(19) United States
(12) Patent Application Publication
(10) Pub. No.: US 2024/0247322 A1
(43) Pub. Date: Jul. 25, 2024

(54) METHODS AND COMPOSITIONS FOR DETECTING SARS-COV2 VIRUS OMICRON, ALPHA, BETA, DELTA, OR GAMMA

(71) Applicant: Universidad de Cartagena, Cartagena (CO)

(72) Inventors: Doris Esther Gómez-Camargo, Cartagena (CO); Steev Orlando Loyola Sosa, Cartagena (CO)

(21) Appl. No.: 18/157,673

(22) Filed: Jan. 20, 2023

(51) Int. Cl. C12Q 1/70

(52) U.S. Cl. CPC C12Q 1/70; C12Q 2600/112; C12Q 2600/156

(57) ABSTRACT

The present invention concerns methods for detecting SARS-CoV 2 virus omicron, alpha, beta, delta and/or gamma as well as kits for performing such methods.

### CROSS REFERENCE TO RELATED SEQUENCE LISTING

This application contains a Sequence Listing submitted via EFS-Web. The entire contents of the text file entitled “MIA0002US_Sequence_Listing.XML” created on Jan. 20, 2023, having a size of 36 kilobytes, is incorporated herein by reference.

### TECHNICAL FIELD

The present invention concerns methods and compositions for detecting SARS-CoV 2 virus omicron, alpha, beta, delta and/or gamma as well as kits for performing such methods.

### BACKGROUND

The severe acute respiratory syndrome-causing type 2 coronavirus (SARS-CoV-2) emerged in China toward the end of 2019, and was subsequently recognized as the causative agent of coronavirus disease (COVID-19) (webpage World Health Organization, Pekar et al. 2021). The rapid spread of the virus globally prompted the World Health Organization (WHO) to declare COVID-19 a pandemic by mid-March 2020 (webpage World Health Organization). As of Aug. 10, 2022, more than 590 million cases and more than 6.4 million deaths from COVID-19 have been reported globally (COVID Live).

SARS-CoV-2 was first sequenced in mid-January 2020 (webpage World Health Organization). Since then, multiple diagnostic tools based on the detection of a genetic region have been developed. The emergence of SARS-CoV-2 variants and the potential impact on the effectiveness of pharmacological and nonpharmacological interventions, transmissibility, immune escape, and disease course generated the need to scale up molecular testing and favour variant typing (webpages SARS CoV 2 classification and Tracking SARS-CoV-2 variants; Meredith et al. 2020). To date, a total of 13 variants recognized by WHO have threatened global public health; Alpha (lineage B.1.1.7 and descendants), Beta (lineage B.1.35 and descendants), Delta (lineage B.1.617.2 and AY), Epsilon (B.1.427 and B.1.429), Eta (B.1.525), Gamma (lineage P.1 and descendants), Iota (B.1.526), Kappa (B.1.617.1), Lambda (C.37), Mu (B.1.621, B.1.621.1), Omicron (lineages B.1.1.529 and BA), Theta (P.3) and Zeta (P.2) (4). (webpage SARS CoV2 classification). However, Alpha, Beta, Gamma, Delta and Omicron have been the only ones described as variants of concern (VOC). In Colombia, the emergence of Mu was described as a variant of concern of SARS-CoV-2 (Laiton-Donato et al. 2021).

Genomic surveillance of SARS-CoV-2 variants relies on the ability of laboratories to sequence whole genomes, identify characteristic mutations throughout the viral genome (website: covariants.org/variants), and through bioinformatics tools assign lineages/clades (website: //clades.nextstrain.org/). Genomic surveillance has been widely promoted as part of strategies designed for early detection of new variants and assessment of geographic spread, and recently for evaluation of vaccine effectiveness in contexts with different viral predominance (Meredith et al. 2020; Riemersma et al. 2021; Stoddard et al. 2021). However, the sequencing capacity deployed globally is heterogeneous, and particularly scarce in low- and middle-income countries (Chen et al. 2022). There is thus a need for diagnosis of SARS CoV 2 variants of concern, which is fast and cost-effective.

### SUMMARY

The present invention describes methods and compositions for detecting one or more of SARS-CoV 2 virus variant(s) that are the omicron, alpha, beta, delta, or gamma variants in a sample. The method is cost effective and can be performed quickly, allowing the identification of the variants omicron, alpha, beta, delta, or gamma in the sample. In turn, the information obtained by the method can be used for such purposes as contemporaneous or preventative treatment of one or more subjects at risk, or can be used to for epidemiological analysis.

In an embodiment, the present invention provides a method for detecting one or more of SARS-CoV 2 virus omicron, alpha, beta, delta, or gamma, in a sample, the method comprising
(i) sequencing a nucleic acid comprising a nucleic acid having at least 80% identity to a nucleic acid corresponding to positions 76 to 645 nucleic acid of SEQ ID NO. 1, wherein the sequenced nucleic acid has a length of at most 1500 nucleotides; and wherein the nucleic acid has been derived from the sample; and
(ii) identifying from the sequenced nucleic acid:
   (iia) one or more nucleotide replacement(s) at positions corresponding to positions 76, 77, 78, 238, 239, 240, 412, 413, 414, 424, 425, 426, 466, 467, 468, 568, 569, 570, 643, 644, and 645 of the nucleic acid as set forth in SEQ ID NO. 1; and/or
   (iib) one or more nucleotide deletion(s) in the sequenced nucleic acid at positions corresponding to positions 205, 206, 207, 208, 209, 210, 430, 431, 432, 469, 470, 471, 472, 473 and 474 of the nucleic acid as set forth in SEQ ID NO. 1; and
(iii) correlating the one or more nucleotide replacement(s) and/or one or more nucleotide deletion(s) with the presence of one or more of SARS-CoV 2 virus omicron, alpha, beta, delta, or gamma, in the sample.

In embodiments, the nucleic acid that is sequenced is complementary DNA (cDNA). Accordingly, the method can also include a step of preparing a composition comprising cDNA which is subjected to sequencing in step (i). Accordingly, the method can also include a step of preparing a composition comprising cDNA, wherein the cDNA-containing composition is derived from SARS-CoV 2 virus RNA, an oligonucleotide primer complementary to the virus RNA, and reverse transcriptase.

The sample having the SARS-CoV 2 virus can be a biological sample, such as one obtained from the subject suspected of having the virus, or can be an environmental sample.

Therefore, the detecting of one or more of SARS-CoV 2 virus omicron, alpha, beta, delta and/or gamma includes diagnosing a subject with one or more of SARS-CoV 2 virus omicron, alpha, beta, delta and/or gamma, wherein the sample is a biological sample that has been obtained from said subject. Additionally or alternatively, the detecting of one or more of SARS-CoV 2 virus omicron, alpha, beta, delta and/or gamma can include the detection of one or more of SARS-CoV 2 virus omicron, alpha, beta, delta and/or gamma in an environmental sample.

The present invention also relates to a kit comprising components for preparing a nucleic acid from a sample that has SARS-CoV 2 virus RNA, wherein the nucleic acid has at least 80% identity to a nucleic acid sequence corresponding to positions 76 to 645 of nucleic acid of SEQ ID NO. 1 and wherein the nucleic acid has a length of at most 1500 nucleotides.

The present method and composition provide benefits for the cost-effective identification of different SARS-CoV-2 variants with short processing and analysis times. This can be beneficial especially in circumstances where diagnostic results are useful for early and effective treatment of subjects that become infected with a SARS-CoV-2 variant. In particular, the present application provides cost-effective sequencing strategies that allow rapid and reliable identification of five different SARS-CoV-2 variants using only one sequencing reaction.

### BRIEF DESCRIPTION OF DRAWINGS

FIG. 1. Graphical representation of the two Sanger sequencing strategies. The figure depicts the complete SARS-CoV-2 genome and the sequencing strategies designed to amplify the gene or part of the spike gene.

FIG. 2. Graphical representation of sequencing strategies for the spike gene and area of coverage according to variant of concern. The map and mutation frequency was obtained from website: //outbreak.info/compare-lineages; and the methods used to generate the map are available at website: //outbreak.info/situation-reports/methods. The mutation map was obtained on 09AGO22, and can be reproduced at; website: //outbreak.info/compare-lineages?pango=Omicron&pango=Alpha&pango=Beta&pango=Delta&pango=Gamma&gene=S&threshold=75&nthresh=1&sub=false&dark=false. The strategies and characteristic mutations of each SARS-CoV-2 variant of concern are presented in the figure.

FIG. 3. Workflow of Sanger sequencing strategies.

FIG. 4. Graphical representation of a Forward and Reverse read for an amplified and sequenced region. Assembly by reference was performed in SeqScape™ Software v3.0 (Manufacturer: Applied Biosystems; Catalog number: 4474978).

FIG. 5. Results and reference mapping of each sequenced segment according to the first and second strategy. The ORF of the spike gene was used as reference for the mapping of each amplified and sequenced segment. For the first sequencing strategy the 12 overlapping segments are shown, and for the second strategy the single amplified segment is shown. Reference assembly was performed in SeqScape™ Software v3.0 (Manufacturer: Applied Biosystems; Catalogue number: 4474978).

FIG. 6. Alignment of primers that can be used to amplify cDNA sequence encoding a part of SARS CoV 2 spike protein (primers/oligonucleotides of SEQ ID NO. 28 and 29).

FIG. 7. Position 20851 to position 23175 corresponding to part of the spike protein of NCBI identifier MN908947 version 3 as of 18 Mar. 2020 are shown. The start codon ATG for the spike protein is underlined and highlighted with bold letters, position 76 to 645 are highlighted with bold letters. Positions of possible forward (fwd) and reverse (rev) primers that could be used in the methods, compositions and kits of the present invention are depicted with respect to SEQ ID NO: 3.

### DETAILED DESCRIPTION

Currently, diagnosis of corona virus variants requires long processing and analysis times. Especially, in a state of emergency, costs and times should be reduced to a maximum. Therefore, the development of sequencing strategies characterized by high throughput, high speed and high reliability are of great utility in such situations. The present application concerns methods that provide for cost-effective sequencing strategies that allow rapid and reliable identification of five different SARS-CoV-2 variants of concern using only one sequencing reaction.

Specifically, the present invention relates to a method for detecting one or more of SARS-CoV 2 virus omicron, alpha, beta, delta and/or gamma in a sample, the method comprising
(i) sequencing a nucleic acid comprising a nucleic acid having at least 80% identity to a nucleic acid corresponding to positions 76 to 645 of the nucleic acid as set forth in SEQ ID NO. 1, wherein the sequenced nucleic acid has a length of at most 1500 nucleotides; and wherein the nucleic acid has been derived from the sample; and
(ii) identifying from the sequenced nucleic acid
   (iia) one or more nucleotide replacement(s) at positions corresponding to positions 76, 77, 78, 238, 239, 240, 412, 413, 414, 424, 425, 426, 466, 467, 468, 568, 569, 570, 643, 644, and 645 of the nucleic acid as set forth in SEQ ID NO. 1; and/or
   (iib) one or more nucleotide deletion(s) in the sequenced nucleic acid at positions corresponding to positions 205, 206, 207, 208, 209, 210, 430, 431, 432, 469, 470, 471, 472, 473 and 474 of the nucleic acid as set forth in SEQ ID NO. 1; and
(iii) correlating the one or more nucleotide replacement(s) and/or one or more nucleotide deletion(s) with the presence of one or more of SARS-CoV 2 virus omicron, alpha, beta, delta and/or gamma.

The method of the present invention can be a method for diagnosing a subject with one or more of SARS-CoV 2 virus omicron, alpha, beta, delta and/or gamma, the method comprising
(i) sequencing a nucleic acid comprising a nucleic acid having at least 80% identity to a nucleic acid corresponding to positions 76 to 645 of the nucleic acid as set forth in SEQ ID NO. 1, wherein the sequenced nucleic acid has a length of at most 1500 nucleotides; and wherein the nucleic acid has been derived from a biological sample that has been obtained from the subject; and
(ii) identifying from the sequenced nucleic acid
   (iia) one or more nucleotide replacement(s) at positions corresponding to positions 76, 77, 78, 238, 239, 240, 412, 413, 414, 424, 425, 426, 466, 467, 468, 568, 569, 570, 643, 644, and 645 of the nucleic acid as set forth in SEQ ID NO. 1; and/or
   (iib) one or more nucleotide deletion(s) in the sequenced nucleic acid at positions corresponding to positions 205, 206, 207, 208, 209, 210, 430, 431, 432, 469, 470, 471, 472, 473 and 474 of the nucleic acid as set forth in SEQ ID NO. 1; and
(iii) diagnosing the subject with SARS-CoV 2 virus omicron, alpha, beta, delta and/or gamma by correlating the one or more nucleotide replacement(s) and/or one or more nucleotide deletion(s) with the presence of one or more of SARS-CoV 2 virus omicron, alpha, beta, delta and/or gamma.

Additionally or alternatively, the method of the present invention is a method for detecting one or more of SARS-CoV 2 virus omicron, alpha, beta, delta and/or gamma in a sample, the method comprising
(i) sequencing a nucleic acid comprising a nucleic acid having at least 80% identity to a nucleic acid corresponding to positions 76 to 645 of the nucleic acid as set forth in SEQ ID NO. 1, wherein the sequenced nucleic acid has a length of at most 1500 nucleotides; and wherein the nucleic acid has been derived from an environmental sample; and
(ii) identifying from the sequenced nucleic acid
   (iia) one or more nucleotide replacement(s) at positions corresponding to positions 76, 77, 78, 238, 239, 240, 412, 413, 414, 424, 425, 426, 466, 467, 468, 568, 569, 570, 643, 644, and 645 of the nucleic acid as set forth in SEQ ID NO. 1; and/or
   (iib) one or more nucleotide deletion(s) in the sequenced nucleic acid at positions corresponding to positions 205, 206, 207, 208, 209, 210, 430, 431, 432, 469, 470, 471, 472, 473 and 474 of the nucleic acid as set forth in SEQ ID NO. 1; and
(iii) correlating the one or more nucleotide replacement(s) and/or one or more nucleotide deletion(s) with the presence of one or more of SARS-CoV 2 virus omicron, alpha, beta, delta and/or gamma.

It is clear to the skilled person that the detection of replacements and/or deletions can be performed on the nucleotide level or on the amino acid level. This means that the skilled person after the sequencing in step (i) can also convert the sequenced nucleic acid into the corresponding amino acid sequence and then analyze the presence of amino acid replacements and amino acid deletions present in that amino acid sequence.

Thus, the present invention also concerns a method for detecting one or more of SARS-CoV 2 virus omicron, alpha, beta, delta and/or gamma in a sample, the method comprising
(i) sequencing a nucleic acid comprising a nucleic acid having at least 80% identity to a nucleic acid corresponding to positions 76 to 645 of the nucleic acid as set forth in SEQ ID NO. 1, wherein the sequenced nucleic acid has a length of at most 1500 nucleotides; and wherein the nucleic acid has been derived from the sample; and (d) converting the sequenced nucleic acid into the corresponding amino acid sequence;
(ii) identifying from the amino acid sequence
   (iia) one or more amino acid replacement(s) at positions corresponding to positions 26, 80, 138, 142, 156, 190 and/or 215 of the amino acid sequence of SEQ ID NO. 2; and/or
   (iib) one or more amino acid deletion(s) at positions corresponding to positions 69, 70, 144, 157 and/or 158 of the amino acid sequence of SEQ ID NO. 2; and
(iii) correlating the one or more amino acid replacement(s) and/or one or more amino acid deletion(s) with the presence of one or more of SARS-CoV 2 virus omicron, alpha, beta, delta and/or gamma.

The present invention also relates to a method for diagnosing a subject with one or more of SARS-CoV 2 virus omicron, alpha, beta, delta and/or gamma, the method comprising
(i) sequencing a nucleic acid comprising a nucleic acid having at least 80% identity to a nucleic acid corresponding to positions 76 to 645 of the nucleic acid as set forth in SEQ ID NO. 1, wherein the sequenced nucleic acid has a length of at most 1500 nucleotides; and wherein the nucleic acid has been derived from a biological sample that has been obtained from the subject; and
(d) converting the sequenced nucleic acid into the corresponding amino acid sequence;
(ii) identifying from the amino acid sequence
   (iia) one or more amino acid replacement(s) at positions corresponding to positions 26, 80, 138, 142, 156, 190 and/or 215 of the amino acid sequence of SEQ ID NO. 2; and/or
   (iib) one or more amino acid deletion(s) at positions corresponding to positions 69, 70, 144, 157 and/or 158 of the amino acid sequence of SEQ ID NO. 2; and
(iii) correlating the one or more amino acid replacement(s) and/or one or more amino acid deletion(s) with the presence of one or more of SARS-CoV 2 virus omicron, alpha, beta, delta and/or gamma.

The present invention also concerns a method for detecting one or more of SARS-CoV 2 virus omicron, alpha, beta, delta and/or gamma in a sample, the method comprising
(i) sequencing a nucleic acid comprising a nucleic acid having at least 80% identity to a nucleic acid corresponding to positions 76 to 645 of the nucleic acid as set forth in SEQ ID NO. 1, wherein the sequenced nucleic acid has a length of at most 1500 nucleotides; and wherein the nucleic acid has been derived from an environmental sample; and
(d) converting the sequenced nucleic acid into the corresponding amino acid sequence;
(ii) identifying from the amino acid sequence
   (iia) one or more amino acid replacement(s) at positions corresponding to positions 26, 80, 138, 142, 156, 190 and/or 215 of the amino acid sequence as set forth in SEQ ID NO. 2; and/or
   (iib) one or more amino acid deletion(s) at positions corresponding to positions 69, 70, 144, 157 and/or 158 of the nucleotide sequence as set forth in SEQ ID NO. 2; and
(iii) correlating the one or more amino acid replacement(s) and/or one or more amino acid deletion(s) with the presence of one or more of SARS-CoV 2 virus omicron, alpha, beta, delta and/or gamma.

In embodiments, the nucleic acid that is sequenced is complementary DNA (cDNA). Accordingly, the method can also include a step of preparing a composition comprising cDNA which is subjected to sequencing in step (i). As such, the method can also include a step of preparing a composition comprising cDNA, wherein the cDNA-containing composition is derived from SARS-CoV 2 virus RNA, one or more oligonucleotide primer complementary to the virus RNA, and reverse transcriptase.

The sample having the SARS-CoV 2 virus can be a biological sample, such as one obtained from the subject suspected of having the virus, or can be an environmental sample.

According to the current disclosure, one or more of SARS CoV 2 variants omicron, alpha, beta, delta and/or gamma are detected using only one sequencing reaction. The person skilled in the art knows which SARS CoV 2 variants are named omicron, alpha, beta, delta and/or gamma and also knows which amino acid replacements and/or deletion these virus variants comprise. Currently known webpages where such variants are described include the webpages CoVariants (website: //covariants.org) outbreak.info SARS-CoV-2 data explorer (website: //outbreak.info/compare-lineages?pango=Omicron&pango=Alpha&pango=Beta&pango=Delta&pango=Gamma&gene=S&threshold=75&nthresh=1&sub=false&dark=false) It is clear that the skilled person can identify the different variants on the nucleotide or on the amino acid level.

The SARS-CoV 2 virus variant omicron can, for example, comprise nucleotide deletions at positions corresponding to positions 205, 206, 207, 208, 209, and 210 of SEQ ID NO. 1 and one or more nucleotide replacement(s) at positions corresponding to positions 424, 425 and/or 426 of SEQ ID NO.1. Additionally or alternatively, the SARS-CoV 2 virus omicron comprises amino acid deletions at positions corresponding to positions 69 and 70 of SEQ ID NO. 2 and an amino acid replacement from G to D at a position corresponding to position 142 of SEQ ID NO. 2.

The term “position” when used herein means the position of a nucleotide or amino acid within a nucleotide or amino acid sequence as described herein. The term “corresponding” as used herein also includes that a certain position is not only determined by the number of the preceding nucleotides or amino acids. The position of a given nucleotide or amino acid in accordance with the present invention, may be substituted/replaced or may vary due to deletions or additional nucleotides/amino acids elsewhere a (sequenced or reference) sequence. The reference sequences used herein are SEQ ID NO. 1 for nucleotides and SEQ ID NO. 2 for amino acids.

Thus, a “corresponding position” preferably means that nucleotides or amino acids may differ in the indicated number but may still have similar neighboring nucleotides or amino acids. Said nucleotides/amino acids which may be exchanged/substituted/replaced deleted or added are also comprised in the term “corresponding position”. Specifically, the skilled person may, when aligning the reference sequence (SEQ ID NO. 1 or SEQ ID NO. 2) with a nucleotide or amino acid sequence of interest, such as e.g., a sequenced nucleic acid that may be additionally converted into the corresponding amino acid sequence as described herein, inspect the sequence of interest when looking for the nucleotide or amino acid positions as specified herein.

In some aspects, the sequence of interests (i.e., bases on a SARS-CoV 2 variant sequence) can be described with reference to its percent identity to all or a portion of the reference sequence (SEQ ID NO. 1 or SEQ ID NO. 2). For example, the sequence of interest comprises a nucleic acid having at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity, to a nucleic acid sequence corresponding to positions 76 to 645 of SEQ ID NO. 1.

In accordance with the present invention, the term “identity” or “percent identity” in the context of the nucleic acid sequence of SEQ ID NO. 1 or the amino acid sequence of SEQ ID No. 2 refers to two or more polynucleotide/polypeptide sequences that are the same or that have a specified percentage of amino acids that are the same (e.g. at least 85%, 90%, 95%, 96%, 97%, 98% or 99% identity) when compared to SEQ ID NO. 1 or 2 and aligned for maximum correspondence over a window of comparison, or over a designated region as measured using a sequence comparison as known in the art or by manual alignment and visual inspection. Sequences having, for example 85% or greater identity are considered to be substantial identical. Those having skill in the art know how to determine percent identity between/among sequences by using, for example, algorithms such as those based on CLUSTALW computer program (Thompsom Nucl. (1994)) or FASTDB (Brutlag et al. (1990)) as known in the art. Also available to those having skill in this art are BLAST and PSI-BLAST algorithms (Altschul et al. (1977)). The BLASTP program for amino acid sequences uses as default a word size (W) of 6, an expect threshold of 0.05, and a comparison of both strands. The BLASTN program for nucleic acid sequences uses as maximal target sequence of 100, an expect threshold of 0.05, a word size of 28, and a comparison of both strands. Furthermore, the BLOSUM 62 scoring matrix (Henikoff and Henikoff (1992)) can be used. For example, BLAST2.6. which stands for Basic Local Alignment Search Tool (Altschul. Nucl. Acids Res. 25 (1997), 3389-3402; Altschul. (1993), 290-300; Altschul et al. (1990), 403-410, can be used to search for local sequence alignments.

As described herein a “nucleotide replacement” also referred to as a “nucleotide substitution” herein means a replacement of a nucleotide relative to a corresponding position of an identified SEQ ID NO. e.g., SEQ ID NO. 1 as disclosed herein. For example, the replacement may be a nucleotide substitution of a nucleotide relative to a position corresponding to position 76 of SEQ ID NO. 1. Such nucleotide replacements to have an effect on the amino acid level need to encode for an amino acid different than the ones defined in SEQ ID NO. 2.

To generate a replacement from the amino acid P to S at a position corresponding to position 26 of SEQ ID NO. 2, the nucleotide triplet present at positions corresponding to positions 76, 77 and 78 of SEQ ID No. 1 needs to be replaced. Specifically, in SEQ ID NO. 1 the nucleotide triplet “C-C-T” at positions 76, 77 and 78 encodes amino acid P (proline) at position 26 of SEQ ID NO. 2. This nucleotide triplet may be replaced by the following nucleotide triplets to encode for amino acid S (serine) at position 26 of SEQ ID NO. 2: “T-C-T”, “T-C-C”, “T-C-A”, “T-C-G”, “A-G-T” or “A-G-C”. Notably, nucleotide replacements leading to a triplet code of “C—C-C”, “C-C-A” or “C-C-G” would be considered silent mutations. This is because all these triplets encode for the amino acid proline and thus, these replacements would not change the amino acid sequence. Thus, following nucleotide replacement(s) may be detected:
from “C” to “T” at a position corresponding to position 76 of SEQ ID NO. 1;
from “C” to “T” at a position corresponding to position 76 of SEQ ID NO. 1 and from “T” to “C” at a position corresponding to position 78 of SEQ ID NO. 1;
from “C” to “T” at a position corresponding to position 76 of SEQ ID NO. 1 and from “T” to “A” at a position corresponding to position 78 of SEQ ID NO. 1;
from “C” to “T” at a position corresponding to position 76 of SEQ ID NO. 1 and from “T” to “G” at a position corresponding to position 78 of SEQ ID NO. 1;
from “C” to “A” at a position corresponding to position 76 of SEQ ID NO. 1 and from “C” to “G” at a position corresponding to position 77 of SEQ ID NO. 1; or
from “C” to “A” at a position corresponding to position 76 of SEQ ID NO. 1, from “C” to “G” at a position corresponding to position 77 of SEQ ID NO. 1 and from “T” to “C” at a position corresponding to position 78 of SEQ ID NO. 1.

To generate a replacement from the amino acid D to A at a position corresponding to position 80 of SEQ ID NO. 2, the nucleotide triplet present at positions corresponding to positions 238, 239 and 240 of SEQ ID NO. 1 needs to be replaced. Specifically, in SEQ ID NO. 1 the nucleotide triplet “G-A-T” at positions 238, 239 and 240 encodes amino acid D (aspartate) at position 80 of SEQ ID NO. 2. This nucleotide triplet may be replaced by the following nucleotide triplets can encode for amino acid A (alanine) at position 80 of SEQ ID No. 2: “G-C-T”, “G-C-C”, “G-C-A”, or “G-C-G”. Notably, nucleotide replacements leading to a triplet code of “G-A-C” would be considered silent mutations. This is because this triplet encodes for the amino acid aspartate and thus, this replacement would not change the amino acid sequence. Thus, following nucleotide replacement(s) may be detected:
from “A” to “C” at a position corresponding to position 239 of SEQ ID NO. 1;
from “A” to “C” at a position corresponding to position 239 of SEQ ID NO. 1 and from “T to “C” at a position corresponding to position 240 of SEQ ID NO. 1;
from “A” to “C” at a position corresponding to position 239 of SEQ ID NO. 1 and from “T” to “A” at a position corresponding to position 240 of SEQ ID NO. 1; or
from “A” to “C” at a position corresponding to position 239 of SEQ ID NO. 1 and from “T” to “G” at a position corresponding to position 240 of SEQ ID No. 1.

To generate a replacement from the amino acid D to Y at a position corresponding to position 138 of SEQ ID NO. 2, the nucleotide triplet present at positions corresponding to positions 412, 413 and 414 of SEQ ID NO. 1 needs to be replaced. Specifically, in SEQ ID NO. 1 the nucleotide triplet “G-A-T” at positions 412, 413 and 414 encodes amino acid D (aspartate) at position 138 of SEQ ID NO. 2. This nucleotide triplet may be replaced by the following nucleotide triplets can encode for amino acid Y (tyrosine) at position 138 of SEQ ID No. 2: “T-A-T” and “T-A-C”. Notably, nucleotide replacements leading to a triplet code of “G-A-C” would be considered silent mutations. This is because this triplet encodes for the amino acid aspartate and thus, this replacement would not change the amino acid sequence. Thus, following nucleotide replacement(s) may be detected:
from “G” to “T” at a position corresponding to position 412 of SEQ ID NO. 1; or
from “G” to “T” at a position corresponding to position 412 of SEQ ID NO. 1 and from “T” to “C” at a position corresponding to position 414 of SEQ ID NO. 1.

To generate a replacement from the amino acid G to D at a position corresponding to position 142 of SEQ ID NO. 2, the nucleotide triplet present at positions corresponding to positions 424, 425 and 426 of SEQ ID NO. 1 needs to be replaced. Specifically, in SEQ ID NO. 1 the nucleotide triplet “G-G-T” at positions 424, 425 and 426 encodes amino acid G (glycine) at position 142 of SEQ ID NO. 2. This nucleotide triplet may be replaced by the following nucleotide triplets that can encode for amino acid D (aspartate) at position 142 of SEQ ID NO. 2: “G-A-T” and “G-A-C”. Notably, nucleotide replacements leading to a triplet code of “G-G-C”, “G-G-A”, or “G-G-G” would be considered silent mutations. This is because this triplet encodes for the amino acid glycine and thus, this replacement would not change the amino acid sequence. Thus, following nucleotide replacement(s) may be detected:
from “G” to “A” at a position corresponding to position 425 of SEQ ID NO. 1; or
from “G” to “A” at a position corresponding to position 425 of SEQ ID NO. 1 and from “T” to “C” at a position corresponding to position 426 of SEQ ID NO. 1.

To generate a replacement from the amino acid E to G at a position corresponding to position 156 of SEQ ID NO. 2, the nucleotide triplet present at positions corresponding to positions 465, 466 and 467 of SEQ ID NO. 1 needs to be replaced. Specifically, in SEQ ID NO. 1 the nucleotide triplet “G-A-G” at positions 465, 466 and 467 encodes amino acid E (glutamate) at position 156 of SEQ ID NO. 2. This nucleotide triplet may be replaced by the following nucleotide triplets that can encode for amino acid G (glycine) at position 142 of SEQ ID NO. 2: “G-G-C”, “G-G-T”, “G-G-A” and “G-G-G”. Notably, nucleotide replacements leading to a triplet code of “G-A-A” would be considered a silent mutation. This is because this triplet encodes for the amino acid glutamate and thus, this replacement would not change the amino acid sequence. Thus, following nucleotide replacement(s) may be detected:
from “A” to “G” at a position corresponding to position 466 of SEQ ID NO. 1 and from “G” to “C” at a position corresponding to position 467 of SEQ ID NO. 1;
from “A” to “G” at a position corresponding to position 466 of SEQ ID NO. 1 and from “G” to “T” at a position corresponding to position 467 of SEQ ID NO. 1;
from “A” to “G” at a position corresponding to position 466 of SEQ ID NO. 1 and from “G” to “A” at a position corresponding to position 467 of SEQ ID NO. 1; or
from “A” to “G” at a position corresponding to position 466 of SEQ ID NO. 1.

To generate a replacement from the amino acid R to S at a position corresponding to position 190 of SEQ ID NO. 2, the nucleotide triplet present at positions corresponding to positions 568, 569 and 570 of SEQ ID NO. 1 needs to be replaced. Specifically, in SEQ ID NO. 1 the nucleotide triplet “A-G-G” at positions 568, 569 and 570 encodes amino acid R (arginine) at position 156 of SEQ ID NO. 2. This nucleotide triplet may be replaced by the following nucleotide triplets that can encode for amino acid S (serine) at position 190 of SEQ ID NO. 2: “A-G-C”, “A-G-T”, “T-C-A”, “T-C-T”, “T-C-C”, or “T-C-G”. Notably, nucleotide replacements leading to a triplet code of “C-G-A”, “C-G-T”, “C-G-C”, “C-G-G”, or “A-G-A” would be considered a silent mutation. This is because this triplet encodes for the amino acid arginine and thus, this replacement would not change the amino acid sequence. Thus, following nucleotide replacement(s) may be detected:
from “G” to “C” at a position corresponding to position 570 of SEQ ID NO. 1;
from “G” to “T” at a position corresponding to position 570 of SEQ ID NO. 1;
from “A” to “T” at a position corresponding to position 568 of SEQ ID NO. 1, from “G” to “C” at a position corresponding to position 569 of SEQ ID No. 1 and from “G” to “A” at a position corresponding to position 570 of SEQ ID NO. 1;
from “A” to “T” at a position corresponding to position 568 of SEQ ID No. 1, from “G” to “C” at a position corresponding to position 569 of SEQ ID NO. 1 and from “G” to “T” at a position corresponding to position 570 of SEQ ID NO. 1;
from “A” to “T” at a position corresponding to position 568 of SEQ ID No. 1, from “G” to “C” at a position corresponding to position 569 of SEQ ID NO. 1 and from “G” to “C” at a position corresponding to position 570 of SEQ ID NO. 1; or
from “A” to “T” at a position corresponding to position 568 of SEQ ID NO. 1 and from “G” to “C” at a position corresponding to position 569 of SEQ ID NO. 1.

To generate a replacement from the amino acid D to G at a position corresponding to position 215 of SEQ ID NO. 2, the nucleotide triplet present at positions corresponding to positions 643, 644 and 645 of SEQ ID NO. 1 needs to be replaced. Specifically, in SEQ ID NO. 1 the nucleotide triplet “G-A-T” at positions 643, 644 and 645 encodes amino acid D (aspartate) at position 215 of SEQ ID NO. 2. This nucleotide triplet may be replaced by the following nucleotide triplets that can encode for amino acid G (glycine) at position 142 of SEQ ID NO. 2: “G-G-C”, “G-G-T”, “G-G-A” and “G-G-G”. Notably, nucleotide replacements leading to a triplet code of “G-A-C” would be considered a silent mutation. This is because this triplet encodes for the amino acid aspartate and thus, this replacement would not change the amino acid sequence. Thus, following nucleotide replacement(s) may be detected:
from “A” to “G” at a position corresponding to position 644 of SEQ ID NO. 1 and from “T” to “C” at a position corresponding to position 645 of SEQ ID NO. 1;
from “A” to “G” at a position corresponding to position 644 of SEQ ID NO. 1;
from “A” to “G” at a position corresponding to position 644 of SEQ ID NO. 1 and from “T” to “A” at a position corresponding to position 645 of SEQ ID NO. 1; or
from “A” to “G” at a position corresponding to position 644 of SEQ ID NO. 1 and from “T” to “G” at a position corresponding to position 645 of SEQ ID NO. 1.

As described herein a “nucleotide deletion” means a deletion of a nucleotide relative to a corresponding position of an identified SEQ ID NO. e.g. SEQ ID NO. 1 as disclosed herein. For example, the deletion may be a nucleotide deletion of a nucleotide relative to a position corresponding to position 205, 206 and 207 of SEQ ID NO. 1.

In some embodiments, there is more than one nucleotide deletion to the nucleic acid, such as a deletion of a nucleotide triplet. Deletion of a nucleotide triplet results in the removal of three consecutive nucleotides otherwise encoding an amino acid. For example, deletion of nucleotides at positions corresponding to position 205, 206, 207, 208, 209, and 210 of SEQ ID NO. 1, would result in removal of amino acids H and V corresponding to positions 69 and 70 (H and V) of SEQ ID NO. 2. A deletion of nucleotides at positions corresponding to position 430, 431, and 432 of SEQ ID NO. 1, would result in removal of amino acid Y corresponding to position 144 (Y) of SEQ ID NO: 2. A deletion of nucleotides at positions corresponding to position 469, 470, 471, 472, 473 and 474 of SEQ ID NO. 1, would result in removal of amino acids F and R corresponding to positions 157 and 158 (F and R) of SEQ ID NO: 2.

As described herein an “amino acid replacement” also referred to as an “amino acid substitution” herein means a replacement of an amino acid relative to a corresponding position of an identified SEQ ID NO. e.g. SEQ ID NO. 2 as disclosed herein. For example, the replacement may be an amino acid substitution of an amino acid relative to a position corresponding to position 26 of SEQ ID NO. 2.

As described herein an “amino acid deletion” means a deletion of an amino acid relative to a corresponding position of an identified SEQ ID NO. e.g., SEQ ID NO. 2 as disclosed herein. For example, the deletion may be an amino acid relative to a position corresponding to position 69 of SEQ ID NO. 2.

The SARS-CoV 2 virus alpha as described herein comprises nucleotide deletions at positions corresponding to positions 205, 206, 207, 208, 209, 210, 430, 431 and 432 of SEQ ID NO. 1. Additionally or alternatively, the SARS-CoV 2 virus alpha comprises amino acid deletions at positions corresponding to positions 69, 70 and 144 of SEQ ID NO. 2.

The SARS-CoV 2 virus beta as described herein comprises one or more nucleotide replacement(s) at positions corresponding to positions 238, 239 and 240 of SEQ ID NO. 1 and one or more nucleotide replacement(s) at positions corresponding to positions 643, 644 and 645 of SEQ ID NO. 1.

For example, the SARS-CoV 2 virus beta as described herein comprises nucleotide replacement(s):
A)
   from “A” to “C” at a position corresponding to position 239 of SEQ ID NO. 1;
   from “A” to “C” at a position corresponding to position 239 of SEQ ID NO. 1 and from “T to “C” at a position corresponding to position 240 of SEQ ID NO. 1;
   from “A” to “C” at a position corresponding to position 239 of SEQ ID NO. 1 and from “T” to “A” at a position corresponding to position 240 of SEQ ID NO. 1; or
   from “A” to “C” at a position corresponding to position 239 of SEQ ID NO. 1 and from “T” to “G” at a position corresponding to position 240 of SEQ ID NO. 1; and
B)
   from “A” to “G” at a position corresponding to position 644 of SEQ ID NO. 1 and from “T” to “C” at a position corresponding to position 645 of SEQ ID NO. 1;
   from “A” to “G” at a position corresponding to position 644 of SEQ ID NO. 1;
   from “A” to “G” at a position corresponding to position 644 of SEQ ID NO. 1 and from “T” to “A” at a position corresponding to position 645 of SEQ ID NO. 1; or
   from “A” to “G” at a position corresponding to position 644 of SEQ ID NO. 1 and from “T” to “G” at a position corresponding to position 645 of SEQ ID NO. 1.

Additionally or alternatively, the SARS-CoV 2 virus beta comprises amino acid replacements from D to A at a position corresponding to position 80 of SEQ ID NO. 2 and from D to G at a position corresponding to position 215 of SEQ ID NO. 2.

The SARS-CoV 2 virus delta as described herein comprises nucleotide deletions at positions corresponding to positions 469, 470, 471, 472, 473, and 474 of SEQ ID NO. 1, one or more nucleotide replacement(s) at position corresponding to position 424, 425 and/or 426 of SEQ ID NO. 1, and one or more nucleotide replacement(s) at positions corresponding to positions 466, 467 and/or 468 of SEQ ID NO. 1.

For example, the SARS-CoV 2 virus delta as described herein comprises nucleotide replacements:
A)
   from “G” to “A” at a position corresponding to position 425 of SEQ ID NO. 1; or
   from “G” to “A” at a position corresponding to position 425 of SEQ ID NO. 1 and from “T” to “C” at a position corresponding to position 426 of SEQ ID NO. 1; and
B)
   from “A” to “G” at a position corresponding to position 466 of SEQ ID NO. 1 and from “G” to “C” at a position corresponding to position 467 of SEQ ID NO. 1;
   from “A” to “G” at a position corresponding to position 466 of SEQ ID NO. 1 and from “G” to “T” at a position corresponding to position 467 of SEQ ID NO. 1;
   from “A” to “G” at a position corresponding to position 466 of SEQ ID NO. 1 and from “G” to “A” at a position corresponding to position 467 of SEQ ID NO. 1; or
   from “A” to “G” at a position corresponding to position 466 of SEQ ID NO. 1.

Additionally or alternatively, the SARS-CoV 2 virus delta comprises amino acid deletions at positions corresponding to positions 157 and 158 of SEQ ID NO. 2, an amino acid replacement from G to D at a position 142 corresponding to position 142 of SEQ ID NO. 2, and an amino acid replacement from E to G at a position corresponding to position 156 of SEQ ID NO. 2.

The SARS-CoV 2 virus gamma as described herein comprises one or more nucleotide replacement(s) at positions corresponding to positions 76, 77 and/or 78 of SEQ ID NO. 1, one or more nucleotide replacement(s) at positions corresponding to position 412, 413 and/or 414 of SEQ ID NO. 1, and one or more nucleotide replacement(s) at positions corresponding to positions 568, 569 and/or 570 of SEQ ID NO. 1.

For example, the SARS-CoV 2 virus gamma as described herein comprises nucleotide replacement(s):
A)
   from “C” to “T” at a position corresponding to position 76 of SEQ ID NO. 1;
   from “C” to “T” at a position corresponding to position 76 of SEQ ID NO. 1 and from “T” to “C” at a position corresponding to position 78 of SEQ ID NO. 1;
   from “C” to “T” at a position corresponding to position 76 of SEQ ID NO. 1 and from “T” to “A” at a position corresponding to position 78 of SEQ ID NO. 1;
   from “C” to “T” at a position corresponding to position 76 of SEQ ID NO. 1 and from “T” to “G” at a position corresponding to position 78 of SEQ ID NO. 1;
   from “C” to “A” at a position corresponding to position 76 of SEQ ID NO. 1 and from “C” to “G” at a position corresponding to position 77 of SEQ ID NO. 1; or
   from “C” to “A” at a position corresponding to position 76 of SEQ ID NO. 1, from “C” to “G” at a position corresponding to position 77 of SEQ ID NO. 1 and from “T” to “C” at a position corresponding to position 78 of SEQ ID NO. 1; and
B)
   from “G” to “T” at a position corresponding to position 412 of SEQ ID NO. 1; or
   from “G” to “T” at a position corresponding to position 412 of SEQ ID NO. 1 and from “T” to “C” at a position corresponding to position 414 of SEQ ID NO. 1; and
C)
   from “G” to “C” at a position corresponding to position 570 of SEQ ID NO. 1;
   from “G” to “T” at a position corresponding to position 570 of SEQ ID NO. 1;
   from “A” to “T” at a position corresponding to position 568 of SEQ ID NO. 1, from “G” to “C” at a position corresponding to position 569 of SEQ ID No. 1 and from “G” to “A” at a position corresponding to position 570 of SEQ ID NO. 1;
   from “A” to “T” at a position corresponding to position 568 of SEQ ID No. 1, from “G” to “C” at a position corresponding to position 569 of SEQ ID NO. 1 and from “G” to “T” at a position corresponding to position 570 of SEQ ID NO. 1;
   from “A” to “T” at a position corresponding to position 568 of SEQ ID No. 1, from “G” to “C” at a position corresponding to position 569 of SEQ ID NO. 1 and from “G” to “C” at a position corresponding to position 570 of SEQ ID NO. 1; or
   from “A” to “T” at a position corresponding to position 568 of SEQ ID NO. 1 and from “G” to “C” at a position corresponding to position 569 of SEQ ID NO. 1.

Additionally or alternatively, the SARS-CoV 2 virus gamma comprises amino acid replacement from P to S at a position corresponding to position 26 of SEQ ID NO. 2, an amino acid replacement from D to Y at a position corresponding to position 138 of SEQ ID NO. 2, and an amino acid replacement from R to S at a position corresponding to position 190 of SEQ ID NO.2.

The SARS-CoV 2 virus omicron as described herein comprises nucleotide deletions at positions corresponding to positions 205, 206, 207, 208, 209, and 210 of SEQ ID NO. 1, and one or more nucleotide replacement(s) at positions corresponding to positions 424, 425 and 426 of SEQ ID NO. 1.

For example, the SARS-CoV 2 virus omicron as described herein comprises nucleotide replacement(s):
from “G” to “A” at a position corresponding to position 425 of SEQ ID NO. 1; or
from “G” to “A” at a position corresponding to position 425 of SEQ ID NO. 1 and from “T” to “C” at a position corresponding to position 426 of SEQ ID NO. 1.

It is clear to the skilled person that the nucleotide/amino acid deletions/replacements as described for each virus variant may include further nucleotide/amino acid deletions/replacements that may develop in the future. Such future mutations and/or deletions are also envisioned by the present invention, as long as they are present in the sequenced sequence of at most 1500 nucleotides that comprises a nucleic acid having at least 80% identity to a nucleic acid corresponding to positions 76 to 645 of the nucleic acid as set forth in SEQ ID NO. 1.

The method of the present invention works for nucleic acids that have been obtained in any type of sample. For example, the sample from which the nucleic acid is obtained is a biological sample or an environmental sample.

Exemplary biological sample include saliva samples, nasal samples, nasopharyngeal samples, sputum samples, oropharyngeal samples, urine samples or blood samples. Thus, the biological sample may be a swab sample. The biological sample may also be a culture supernatant obtained from a culture of a biological sample.

It is envisioned that the nucleic acid has been derived from a biological sample that has been obtained from the subject. The subject may be any subject of interest. The subject may be a human or an animal. The subject may be a mammal such as a human being, cat, dog, horse, sheep, camel, cow, ape, pig or goat. Preferably the subject is a human. The subject may also be a human patient.

The sample from which the nucleic acid is obtained may also be an environmental sample. The environmental sample may be any environmental sample. Exemplary environmental samples include sewage water, or samples taken from air or surface.

The term “natural” refers to a composition that is found in nature. In a natural sample there is no human manipulation of the composition. Conversely, the term “non-natural” refers to a composition or component that is not found in nature, such as one that requires human manipulation in some form. Various non-natural compositions and components are described herein, including, but not limited to synthetically prepared oligonucleotides, cDNA, PCR products, and compositions including any of these components, such as RT reaction compositions, PCR reaction compositions, and nucleic acid sequencing compositions. Non-natural compositions also include biological or environmental samples that have been modified or treated in some way, such as by enrichment or purification.

The methods of the present invention contemplate that a nucleic acid that has been derived from a sample is sequenced.

As used herein a “a nucleic acid that has been derived from a sample” such as a biological sample or environmental sample refers to any suitable nucleic acid derivable from said sample. For example, the sample may comprise SARS CoV 2 viral RNA. A nucleic acid is derived from this sample, when converting the SARS CoV 2 viral RNA into the corresponding cDNA. The corresponding cDNA may be obtained by any suitable technique or as elsewhere disclosed herein. The corresponding cDNA may e.g. be obtained by reverse transcription of the SARS CoV 2 viral RNA present in said sample.

The sequencing includes sequencing of a nucleic acid having at least 80% identity to a nucleic acid comprising a nucleic acid corresponding to positions 76 to 645 of the nucleic acid as set forth in SEQ ID NO. 1, wherein the sequenced nucleic acid has a length of at most 1500 nucleotides.

In principle, any sequence technique can be used for these purposes. Such sequence techniques are known to the skilled person. Some sequencing techniques are for example summarized in Ozsolak and Milos (2011) “RNA sequencing: advances, challenges and opportunities” Nat Rev Genet. 2011 February; 12(2): 87-98 and Klerk et al. (2014) “RNA sequencing: from tag-based profiling to resolving complete transcript structure” Cell. Mol. Life Sci. (2014) 71:3537-3551. For example, the sequencing may be performed by Sanger sequencing, Maxam-Gilbert sequencing, shotgun sequencing, Ion Torrent sequencing, pyrosequencing, sequencing by synthesis, Single Molecule Real-Time (SMRT) sequencing or nanopore sequencing. Sequencing may also be performed by a method as described in Paden et al. (2020) “Rapid, Sensitive, Full-Genome Sequencing of Severe Acute Respiratory Syndrome Coronavirus 2” Emerging Infectious Diseases Vol. 26, No. 10, pp. 2401-2405; Sanger et al. (1977) “DNA sequencing with chain-terminating inhibitors” Proc. Natl. Acad. Sci. USA Vol. 74, No. 12, pp. 5463-5467, or by a method as described in the examples.

As defined herein the sequenced nucleic acid should comprise a sequence having at least 80% identity to a nucleic acid corresponding to positions 76 to 645 of the nucleic acid as set forth in SEQ ID NO. 1. Thus, the sequenced sequence is a cDNA sequence.

A “cDNA” as used herein refers to a cDNA that has been reversely transcribed from the corresponding RNA. Notably, the SARS-CoV-2 has a linear, positive-sense, single-stranded RNA genome that is about 30,000 bases long. This RNA or parts of it may be reversely transcribed into the corresponding cDNA.

A “reverse transcription” as used herein means the process of the generation of complementary DNA (cDNA) from an RNA template usually mediated by an enzyme such as the reverse transcriptase. Well studied reverse transcriptases include: HIV-reverse transcriptase from human immunodeficiency virus type 1 (PDB 1 HMV), M-MLV reverse transcriptase from the Moloney murine leukemia virus and the AMV reverse transcriptase from avian myeloblastosis virus. However, since the cDNA is generated from viral RNA that may slightly differ in different viruses, it is clear that the cDNA as disclosed in SEQ ID NO. 1 refers to the actual consensus cDNA corresponding to the spike protein used to describe the original SARS-CoV 2 virus.

As defined herein the sequenced nucleic acid should have a length of at most 1500 nucleotides. It is clear to the skilled person that the mentioned length is a theoretical size. It is specifically theoretical, since the sequence as defined in SEQ ID NO. 1 concerns a consensus cDNA corresponding to the spike protein of SARS CoV 2. Notably, the calculation of the length of the sequenced sequence does not include possible deletions or insertions, and excludes the region of primer annealing (both forward and reverse). It is also envisioned that the sequenced nucleic acid has a length of at most 1400 nucleotides, at most 1300 nucleotides, at most 1200 nucleotides, at most 1100 nucleotides, at most 1000 nucleotides, at most 900 nucleotides, at most 800 nucleotides, at most 700 nucleotides or at most 600 nucleotides or less. The person skilled in the art knows how to calculate the length of a given nucleic acid.

The methods of the present invention as disclosed herein may comprise a series of additional method steps. For example, the methods may further comprise a step (a) extracting RNA from a sample.

The sample can be any sample or a sample as disclosed elsewhere herein. The extracted RNA refers to the RNA of the SARS-CoV 2 virus that corresponds to a sequence that includes a sequence as set forth in SEQ ID NO. 1 or parts thereof. The extraction of RNA is the purification of RNA from the sample as disclosed elsewhere herein. Methods for RNA extraction are known to the skilled person. Any RNA extraction method may be used in the methods disclosed herein. Such methods are inter alia described in Deng et al. (2005) “Comparison of six RNA extraction methods for the detection of classical swine fever virus by real-time and conventional reverse transcription-PCR” J Vet Diagn Invest 17:574-578 or Wozniak et al. (2020) “A simple RNA preparation method for SARS-CoV-2 detection by RT-qPCR” Scientific Reports:16608 or methods as described in the examples. For example, the extracting of the RNA is performed using organic extraction, preferably phenol-Guanidine Isothiocyanate (GITC)-based solutions, silica-membrane based spin column technology, or paramagnetic particle technology.

RNA extraction can provide a non-natural composition wherein RNA is enriched, partially purified, or substantially purified. The RNA extraction process can separate RNA therein from other components present in the sample it is obtained from, such as non-RNA biological material from a subject or patient sample or an environmental sample. A composition with extracted RNA can optionally include other biological materials (e.g., proteins, lipids, carbohydrates, other nucleic acids), but generally not in an amount that would interfere with subsequent reactions using oligonucleotides such as reverse transcription or PCR.

It is clear that the extracted RNA sequence(s) as referred to herein e.g. in step (a) relate to RNA sequences obtained from SARS CoV 2 virus or parts thereof. The extraction can include the whole (RNA) genome of the SARS CoV 2 virus or parts thereof, e.g. only parts that include the region corresponding to the region encoding for the spike protein of the SARS CoV 2 virus.

The methods disclosed herein may further comprise a step (b) converting the RNA nucleic acid into the corresponding cDNA sequence. As used herein a “cDNA” DNA that is complementary to SARS CoV 2 RNA. cDNA is usually obtained by reverse transcription as elsewhere described herein. cDNA as described herein can be single (first strand) or double stranded (first and second strand).

First-strand synthesis of cDNA can utilize either specific oligonucleotides (primers), random primers, or a combination of these strategies to prime the reverse transcription reaction. Priming a reaction with specific oligonucleotides initiates the synthesis at a specific position. Random primers are oligonucleotide sequences of six or more bases. Each random primer has a different arrangement of bases, giving it the potential to anneal at many random points on an RNA transcript, ensuring complete coverage of the transcript. Roughly, for cDNA generation dNTPs (dGTP, dCTP, dATP and dTTP), primers/oligonucleotides, and reverse transcriptase are needed. The sample comprising RNA is contacted with dNTPs, primers and reverse transcriptase under suitable conditions to allow for DNA synthesis (first strand synthesis). Then the RNA is removed and optionally the second strand of cDNA can be synthesized.

As used herein the “corresponding cDNA sequence” refers to the cDNA sequence that is complementary to the indicated RNA sequence. The term “complementary” refers to the natural binding of polynucleotides under permissive salt and temperature conditions by base-pairing. For example, the sequence “A-G-C” binds the complementary sequence “T-C-G”. Since in RNA sequences the T (thymine) is present as an uracil (U), it is clear that when an RNA is converted into the corresponding cDNA that any “U” in the RNA is converted into an “A”, while any “A” in the RNA is converted into a “T” in the cDNA sequence. Typically, the conversion is performed by reverse transcription as described elsewhere herein.

Methods for converting RNA into the corresponding cDNA are known to the skilled person. Any suitable technique may be used in the methods as described herein. Some suitable methods are inter alia described in Paden et al. 2020, Matranga et al. (2016) Unbiased Deep Sequencing of RNA Viruses from Clinical Samples” Journal of Visualized Experiments, 113, e54117, pp. 1-9 or a method as described in the examples. The converting of the RNA nucleic acid into the corresponding cDNA sequence can for example be performed by reverse transcription such as RT-PCR, poly-A tagging, or RNase H-DNA polymerase I-mediated second-strand cDNA synthesis.

Accordingly, the disclosure also provides compositions comprising SARS CoV 2 RNA, dNTPs, one or more oligonucleotides that are complementary to SARS CoV 2 RNA, and reverse transcriptase (RT). RT reaction compositions also typically include reaction buffer components such as Tris-HCl, KCl, MgCl2, spermidine, and dithiothreitol (DTT).

The one or more oligonucleotides that are complementary to SARS CoV 2 RNA can be any suitable oligonucleotide. As noted, exemplary lengths for oligonucleotides are in the range of about 10 to 50, about 10-35, about 10-25, about 15-35, or about 10-20 nucleic acid monomer units. In embodiments, the oligonucleotide is complementary to the SARS CoV 2 RNA, and has a sequence that is based on a nucleic acid sequence found between position 1 and position 787, or more preferably between position 70 and position 627, or even more preferably between position 131 and position 139, or even more preferably between position 139 and position 596 of SEQ ID NO: 3 (e.g. for forward primers). Additionally or alternatively, the oligonucleotide is complementary to the SARS CoV 2 RNA, and has a sequence that is based on a nucleic acid sequence found between position 1358 and position 2299, or more preferably between position 1388 and position 2278, or even more preferably between position 1473 and position 2278 or even more preferably between position 1473 and position 2325 of SEQ ID NO: 3 (e.g. for reverse primers). As such, in embodiments the oligonucleotide that is complementary to the SARS CoV 2 RNA, and has a sequence that is based on a nucleic acid sequence of SEQ ID NO: 3 can have sequence of any one of SEQ ID NO. 4, 6 (or 28), 9 (or 29), 11, 13, 30, 31 or 32.

For example, two oligonucleotides that are complementary to SARS CoV 2 RNA can be used. It is also contemplated that e.g., one oligonucleotide that is complementary to the SARS CoV 2 RNA, and has a sequence that is based on a nucleic acid sequence found between position 1 and position 787 is used in combination with one oligonucleotide that is complementary to the SARS CoV 2 RNA, and has a sequence that is based on a nucleic acid sequence found between position 1358 and position 2299. For example, the first oligonucleotide may be selected from any one of the oligonucleotides of SEQ ID NO. 4, 6 (or 28), 30 or 31 and the second oligonucleotide can be selected from any one of the oligonucleotides of SEQ ID NO. 9 (or 29), 11, 13 or 32.

It is also contemplated that the methods as disclosed herein further comprise step (c) amplifying the cDNA comprising a sequence corresponding to positions 76 to 645 of the nucleic acid as set forth in SEQ ID NO. 1 (cDNA), wherein the amplified cDNA sequence has a length of at most 1500 nucleotides.

The skilled person knows how to amplify cDNA sequences. For example, the amplification of the cDNA is performed by PCR. Thus, the converting of the RNA nucleic acid into the corresponding cDNA sequence and the amplification of the cDNA can be performed by RT-PCR. For example, the skilled person may use a method as disclosed in the examples to amplify the cDNA of interest.

Accordingly, the disclosure also provides compositions comprising SARS CoV 2 cDNA, dNTPs, at least two oligonucleotides that are complementary to SARS CoV 2 DNA, and a thermostable DNA polymerase, such as Taq polymerase. PCR reaction compositions can also include reaction buffer components such as Tris-HCl, KCl, MgCl2, spermidine, and dithiothreitol (DTT), similar to the RT reaction. The two oligonucleotides (forward primer, reverse primer) that are complementary to SARS CoV 2 RNA, independently have lengths in the range of about 10 to 50, about 10-35, about 15-35, about 10-25, or about 10-20 nucleic acid monomer units. In embodiments, the forward oligonucleotide is complementary to the SARS CoV 2 DNA, and has a sequence that is based on a nucleic acid sequence found between position 1 and position 787, or more preferably between position 70 and position 627, or more preferably between position 131 and position 606 or more preferably between position 139 and position 596, of SEQ ID NO: 3, and the reverse oligonucleotide is complementary to the SARS CoV 2 DNA, and has a sequence that is based on a nucleic acid sequence found between position 1358 and position 2299, or more preferably between position 1388 and position 2278, or more preferably between position 1473 and position 2278, or more preferably between position 1473 and position 2325 of SEQ ID NO: 3. For example, the forward oligonucleotide may be selected from any one of the oligonucleotides of SEQ ID NO. 4, 6 (or 28), 30 or 31 and the reverse oligonucleotide can be selected from any one of the oligonucleotides of SEQ ID NO. 9 (or 29), 11, 13 or 32.

It is envisioned that the nucleic acid that is sequenced in step (i) is the cDNA obtained in step (c).

It is also envisioned that the sequencing of step (i) and/or the amplification of step (c) can comprise the use of an oligonucleotide of SEQ ID NO. 28 and an oligonucleotide of SEQ ID NO. 29.

As used herein the term “oligonucleotide” refers to a nucleic acid that includes at least two nucleic acid monomer units, more typically from 5 to 175 nucleic monomer units, more typically from 8 to 100 nucleic monomer units, and still more typically from 10 to 50 nucleic acid monomer units (e.g., about 15, about 20, about 25, about 30, about 35 or more nucleic acid monomer units). The exact size of an oligonucleotide will depend on many factors including the function or use of the oligonucleotide. Oligonucleotides can be prepared by any suitable method. For example, an oligonucleotide may be obtained by isolation of an existing or natural sequence, cloning of appropriate sequence or by direct chemical synthesis. An oligonucleotide as described herein may be a primer.

A “primer” as used herein refers to an oligonucleotide capable of acting as a point of initiation of template-directed nucleic acid synthesis when placed under conditions in which polynucleotide extension is initiated (e.g. under conditions comprising the presence of requisite nucleosides triphosphates (as dictated by the template that is copied) and a polymerase in an appropriate buffer and at suitable temperature of cycle(s) of temperatures (e.g. as in polymerase chain reaction (PCR). A primer is typically a single stranded oligonucleotide (e.g. oligodeoxyribose). The appropriate length of a primer depends on the intended use of the primer but typically ranges from 6 to 40 nucleotides, more typically from 15 to 35 nucleotides. Short primer molecules generally require lower temperatures to form sufficiently stable hybrid complexeswith the target sequence of a template than longer primers.

A primer can be labelled, if desired, by incorpbrating a label detectable by spectroscopic, photochemical, biochemical, immunochemical, or chemical means. For example, useful labels include ³²P, flourescent dyes, electron-dense reagents, enzymes (as commonly used in ELISA assays), biotin, or haptens and proteins for which antisera or monoclonal antibodies are available.

It is also contemplated that the methods disclosed herein comprise a step of purification of PCR products, e.g., of the obtained amplified cDNA PCR products. Methods how such purification can be performed are known to the skilled person and exemplarily described in the examples. For example, purification of the cDNA can be performed by gel electrophoresis or enzymatic purification (as disclosed in the examples).

It is further contemplated that the detecting of the presence or absence of one or more nucleotide replacement(s) and/or one or more deletion(s) in the sequenced nucleic acid is performed by comparing the sequenced nucleic acid to the nucleic acid corresponding to positions 76 to 645 as set forth in SEQ ID NO. 1 in the methods as disclosed herein. Thus, the methods as disclosed herein may further comprise a step (e) comparing the sequenced nucleic acid to the nucleic acid of positions 76 to 645 as set forth in SEQ ID NO. 1.

It is also envisioned that the methods disclosed herein can further comprise a step (d) converting the sequenced nucleic acid into the corresponding amino acid sequence. The corresponding amino acid sequence can comprise a sequence corresponding to positions 26 to 215 of the sequence as set forth in SEQ ID NO. 2.

Therefore, the detecting of the presence or absence of one or more amino acid replacement(s) and/or one or more amino acid deletion(s) can be performed by comparing the obtained amino acid sequence to the amino acid sequence of positions 26 to 215 as set forth in SEQ ID NO. 2 in the methods as disclosed herein. More particularly, the detected one or more replacements and/or one or more deletions in the obtained amino acid can correspond to one or more amino acid replacement(s) corresponding to positions 26, 80, 138, 142, 156, 190 and/or 215 and one or more amino acid deletion(s) corresponding to positions 69, 70, 144, 157 and/or 158 of the amino acid sequence set forth in SEQ ID NO. 2.

Thus, the methods as disclosed herein may further comprise a step (e) comparing the sequenced amino acid sequence to the amino acid sequence of positions 26 to 215 as set forth in SEQ ID NO. 2.

The method disclosed herein may thus comprise a further step of (f1) correlating the one or more nucleotide replacement(s) and/or one or more nucleotide deletion(s) with and/or diagnosing a subject with one or more of SARS-CoV 2 virus omicron, alpha, beta, delta and/or gamma by correlating the one or more nucleotide replacement(s) and/or one or more nucleotide deletion(s) with
(x) omicron, when nucleotide deletions at positions corresponding to positions 205, 206, 207, 208, 209, and 210 of SEQ ID NO. 1 and one or more nucleotide replacement(s) at positions corresponding to positions 424, 425 and/or 426 of SEQ ID NO.1 are detected;
(xi) alpha, when nucleotide deletions at positions corresponding to positions 205, 206, 207, 208, 209, 210, 430, 431 and 432 of SEQ ID NO. 1 are detected; (xii) beta, when one or more nucleotide replacement(s) at positions corresponding to positions 238, 239 and 240 of SEQ ID NO. 1 and one or more nucleotide replacement(s) at positions corresponding to positions 643, 644 and 645 of SEQ ID NO. 1 are detected;
(xiii) delta, when nucleotide deletions at positions corresponding to positions 469, 470, 471, 472, 473, and 474 of SEQ ID NO. 1, one or more nucleotide replacements at position corresponding to position 424, 425 and/or 426 of SEQ ID NO. 1, and one or more nucleotide replacement(s) at positions corresponding to positions 466, 467 and/or 468 of SEQ ID NO. 1 are detected; and/or
(xiv) gamma, when one or more nucleotide replacement(s) at positions corresponding to positions 76, 77 and/or 78 of SEQ ID NO. 1, one or more nucleotide replacement(s) at positions corresponding to position 412, 413 and/or 414 of SEQ ID NO. 1, and one or more nucleotide replacement(s) at positions corresponding to positions 568, 569 and/or 570 of SEQ ID NO.1 are detected.

Additionally or alternatively, the methods as disclosed herein may comprise a step of
(f2) correlating the one or more amino acid replacement(s) and/or one or more amino acid deletion(s) with and/or diagnosing a subject with one or more of SARS-CoV 2 virus omicron, alpha, beta, delta and/or gamma by correlating the one or more amino acid replacement(s) and/or one or more amino acid deletion(s) with
   (x) omicron, when amino acid deletions at positions corresponding to positions 69 and 70 of SEQ ID NO. 2 and an amino acid replacement from G to D at a position corresponding to position 142 of SEQ ID NO. 2 are detected;
   (xi) alpha, when amino acid deletions at positions corresponding to positions 69, 70 and 144 of SEQ ID NO. 2 are detected;
   (xii) beta, when amino acid replacements from D to A at a position corresponding to position 80 of SEQ ID No. 2 and from D to G at a position corresponding to positions 215 of SEQ ID NO. 2 are detected;
   (xiii) delta, when amino acid deletions at positions corresponding to positions 157 and 158 of SEQ ID NO. 2, an amino acid replacement from G to D at a position corresponding to position 142 of SEQ ID NO. 2, and an amino acid replacement from E to G at a position corresponding to position 156 of SEQ ID NO. 2 are detected; and/or
   (xiv) gamma, when an amino acid replacement from P to S at a position corresponding to position 26 of SEQ ID NO. 2, an amino acid replacement from D to Y at a position corresponding to position 138 of SEQ ID NO. 2, and an amino acid replacement from R to S at a position corresponding to position 190 of SEQ ID NO. 2 are detected.

In view of the different method steps discussed herein, the methods disclosed herein may comprise
(a) extracting RNA from a sample;
(b) converting the RNA into cDNA;
(c) amplifying the cDNA having at least 80% identity to a nucleic acid corresponding to positions 76 to 645 of the nucleic acid as set forth in SEQ ID NO. 1, wherein the amplified cDNA has a length of at most 1500 nucleotides;
(i) sequencing the amplified cDNA;
(e) comparing the sequenced cDNA to positions corresponding to 76 to 645 of SEQ ID NO. 1;
(ii) identifying from the sequenced cDNA
   (iia) one or more nucleotide replacement(s) at positions corresponding to positions 76, 77, 78, 238, 239, 240, 412, 413, 414, 424, 425, 426, 466, 467, 468, 568, 569, 570, 643, 644, and 645 of the nucleic acid encoding the spike protein of the SARS-CoV2 virus as set forth in SEQ ID NO. 1; and/or
   (iib) one or more nucleotide deletion(s) in the sequenced nucleic acid at positions corresponding to positions 205, 206, 207, 208, 209, 210, 430, 431, 432, 469, 470, 471, 472, 473, 474 of the nucleic acid encoding the spike protein of the SARS-CoV2 virus as set forth in SEQ ID NO. 1; and
(f) correlating the one or more nucleotide replacement(s) and/or one or more nucleotide deletion(s) with
   (x) omicron, when nucleotide deletions at positions corresponding to positions 205, 206, 207, 208, 209, and 210 of SEQ ID NO. 1 and one or more nucleotide replacements at positions corresponding to positions 424, 425 and/or 426 of SEQ ID NO.1 are detected;
   (xi) alpha, when nucleotide deletions at positions corresponding to positions 205, 206, 207, 208, 209, 210, 430, 431 and 432 of SEQ ID NO. 1 are detected;
   (xii) beta, when one or more nucleotide replacement(s) at positions corresponding to positions 238, 239 and 240 of SEQ ID NO. 1 and one or more nucleotide replacements at positions corresponding to positions 643, 644 and 645 of SEQ ID NO. 1 are detected;
   (xiii) delta, when nucleotide deletions at positions corresponding to positions 469, 470, 471, 472, 473, and 474 of SEQ ID NO. 1, one or more nucleotide replacements at positions corresponding to position 424, 425 and/or 426 of SEQ ID NO. 1, and one or more nucleotide replacement(s) at positions corresponding to positions 466, 467 and/or 468 of SEQ ID NO. 1 are detected; and/or
   (xiv) gamma, when one or more nucleotide replacement(s) at positions corresponding to positions 76, 77 and/or 78 of SEQ ID NO. 1, one or more nucleotide replacement(s) at positions corresponding to position 412, 413 and/or 414 of SEQ ID NO. 1, and one or more nucleotide replacement(s) at positions corresponding to positions 568, 569 and/or 570 of SEQ ID NO.1 are detected.

It is envisioned that the methods disclosed herein may further comprise a step
(e) comparing the sequenced cDNA sequence to the sequence of positions 26 to 215 of SEQ ID NO. 2. It is clear that positions 26 and 215 are included in said sequence.

Therefore, the methods disclosed herein may comprise
(a) extracting RNA from a sample;
(b) converting the RNA into cDNA;
(c) amplifying cDNA comprising a cDNA nucleic acid having at least 80% identity to a nucleic acid corresponding to positions 76 to 645 of the nucleic acid as set forth in SEQ ID NO. 1, wherein the cDNA has a length of at most 1500 nucleotides;
(i) sequencing the amplified cDNA;
(d) converting the sequenced cDNA into the corresponding amino acid sequence; (e) comparing the amino acid sequence to the sequence of positions 26 to 215 of SEQ ID NO. 2;
(ii) identifying from the amino acid sequence
   (iia) one or more amino acid replacement(s) at positions corresponding to positions 26, 80, 138, 142, 156, 190 and/or 215 of the amino acid sequence of as set forth in SEQ ID NO. 2; and/or
   (iib) one or more amino acid deletion(s) at positions corresponding to positions 69, 70, 144, 157 and/or 158 of the protein sequence as set forth in SEQ ID NO. 2; and
(f) correlating the one or more amino acid replacement(s) and/or one or more amino acid deletion(s) with
   (x) omicron, when amino acid deletions at positions corresponding to positions 69 and 70 of SEQ ID NO. 2 and an amino acid replacement from G to D at a position corresponding to position 142 of SEQ ID NO.2 are detected;
   (xi) alpha, when amino acid deletions at positions corresponding to positions 69, 70 and 144 of SEQ ID NO. 2 are detected;
   (xii) beta, when amino acid replacements from D to A at a position corresponding to position 80 of SEQ ID No. 2 and from D to G at a position corresponding to positions 215 of SEQ ID NO. 2 are detected;
   (xiii) delta, when amino acid deletions at positions corresponding to positions 157 and 158 of SEQ ID NO. 2, an amino acid replacement from G to D at a position corresponding to position 142 of SEQ ID NO. 2, and an amino acid replacement from E to G at a position corresponding to position 156 of SEQ ID NO. 2 are detected; and/or
   (xiv) gamma, when an amino acid replacement from P to S at a position corresponding to position 26 of SEQ ID NO. 2, an amino acid replacement from D to Y at a position corresponding to position 138 of SEQ ID NO. 2, and an amino acid replacement from R to S at a position corresponding to position 190 of SEQ ID NO. 2 are detected.

Notably, the PCR products of the cDNA obtained in step (c) and/or the sequenced cDNA products of step (i) may be purified e.g. by electrophoresis or by enzymatic purification as described in the examples.

The methods as disclosed herein may further comprise a step
(g) treating the subject diagnosed with SARS-CoV 2 virus omicron, alpha, beta, delta and/or gamma.

A “treatment”, “treat” or “therapy” as used herein refers to alleviation, attenuation (e.g. of progression) of SARS CoV 2 infection. Treatment may include that the subject diagnosed with SARS-CoV 2 virus omicron, alpha, beta, delta and/or gamma takes an antiviral drug. Such an antiviral drug may, for example, be paxlovid, lagevrio (molnupiravir) and/or veklury@ (remdesivir). Treatment may also just include social isolation of the subject diagnosed with SARS-CoV 2 virus omicron, alpha, beta, delta and/or gamma to not infect further subjects.

The present invention also relates to a kit comprising means for sequencing a nucleic acid comprising a nucleic acid having at least 80% identity to a nucleic acid corresponding to positions 76 to 645 of the nucleic acid as set forth in SEQ ID NO. 1; wherein the sequenced nucleic acid has a length of at most 1500 nucleotides.

The present invention also relates to a kit comprising components for preparing a nucleic acid from a sample that has SARS-CoV 2 virus RNA, wherein the nucleic acid has at least 80% identity to a nucleic acid corresponding to positions 76 to 645 of nucleic acid of SEQ ID NO. 1; wherein the nucleic acid has a length of at most 1500 nucleotides.

The means or the kit may comprise an oligonucleotide of SEQ ID NO. 28 and an oligonucleotide of SEQ ID NO. 29, dNTPs, reverse transcriptase, DNA polymerase, and/or buffers.

Throughout this specification and the claims which follow, unless the context requires otherwise, the word “comprise”, and variations such as “comprises” and “comprising” will be understood to imply the inclusion of a stated integer or step. When used herein the term comprising can be substituted with the term “containing” or “including” or “having”. When used herein “consisting of excludes any element or step or ingredient not specified.

The term “including” means “including but not limited to”, which terms can be used interchangeably.

It should be understood that this invention is not limited to the particular methodology, protocols, material, reagents and substances etc. described herein but can vary. The terminology used herein is for the purpose of describing particular embodiments only, and is not intended to limit the scope of the invention which is limited solely by the scope of the claims.

All publications cited throughout the text of this specification (including all patents, patent applications, scientific publications, instructions, etc. whether supra or infra are hereby incorporated by reference in their entirety. Nothing herein is to be construed as an admission that the invention is not entitled antedate such disclosure by virtue of prior invention. To the extend the material incorporated by reference contradicts or is inconsistent with this specification, the specification will supersede any such material.

The content of all documents cited herein is incorporated by reference in their entirety.

The present invention is further characterized by the following items:
1. A method for detecting one or more of SARS-CoV 2 virus omicron, alpha, beta, delta and/or gamma in a sample, the method comprising
(i) sequencing a nucleic acid comprising a nucleic acid having at least 80% identity to a nucleic acid corresponding to positions 76 to 645 of the nucleic acid as set forth in SEQ ID NO. 1 (cDNA sequence), wherein the sequenced nucleic acid has a length of at most 1500 nucleotides; and wherein the nucleic acid has been derived from the sample; and
(ii) identifying from the sequenced nucleic acid
   (iia) one or more nucleotide replacement(s) at positions corresponding to positions 76, 77, 78, 238, 239, 240, 412, 413, 414, 424, 425, 426, 466, 467, 468, 568, 569, 570, 643, 644, and 645 of the nucleic acid as set forth in SEQ ID NO. 1; and/or
   (iib) one or more nucleotide deletion(s) in the sequenced nucleic acid at positions corresponding to positions 205, 206, 207, 208, 209, 210, 430, 431, 432, 469, 470, 471, 472, 473 and 474 of the nucleic acid as set forth in SEQ ID NO. 1; and
(iii) correlating the one or more nucleotide replacement(s) and/or one or more nucleotide deletion(s) with the presence of one or more of SARS-CoV 2 virus omicron, alpha, beta, delta and/or gamma.

2. The method of item 1, wherein the sample is a biological sample or an environmental sample.

3. A method for diagnosing a subject with one or more of SARS-CoV 2 virus omicron, alpha, beta, delta and/or gamma, the method comprising
(i) sequencing a nucleic acid comprising a nucleic acid having at least 80% identity to a nucleic acid corresponding to positions 76 to 645 of the nucleic acid as set forth in SEQ ID NO. 1, wherein the sequenced nucleic acid has a length of at most 1500 nucleotides and wherein the nucleic acid has been derived from a biological sample that has been obtained from the subject; and
(ii) identifying from the sequenced nucleic acid
   (iia) one or more nucleotide replacement(s) at positions corresponding to positions 76, 77, 78, 238, 239, 240, 412, 413, 414, 424, 425, 426, 466, 467, 468, 568, 569, 570, 643, 644, and 645 of the nucleic acid as set forth in SEQ ID NO. 1; and/or
   (iib) one or more nucleotide deletion(s) in the sequenced nucleic acid at positions corresponding to positions 205, 206, 207, 208, 209,210, 430, 431, 432, 469, 470, 471, 472, 473 and 474 of the nucleic acid as set forth in SEQ ID NO. 1; and
(iii) diagnosing the subject with SARS-CoV 2 virus omicron, alpha, beta, delta and/or gamma by correlating the one or more nucleotide replacement(s) and/or one or more nucleotide deletion(s) with the presence of one or more of SARS-CoV 2 virus omicron, alpha, beta, delta and/or gamma.

4. A method for detecting one or more of SARS-CoV 2 virus omicron, alpha, beta, delta and/or gamma in a sample, the method comprising
(i) sequencing a nucleic acid comprising a nucleic acid having at least 80% identity to a nucleic acid corresponding to positions 76 to 645 of the nucleic acid as set forth in SEQ ID NO. 1, wherein the sequenced nucleic acid has a length of at most 1500 nucleotides; and wherein the nucleic acid has been derived from an environmental sample; and
(ii) identifying from the sequenced nucleic acid
   (iia) one or more nucleotide replacement(s) at positions corresponding to positions 76, 77, 78, 238, 239, 240, 412, 413, 414, 424, 425, 426, 466, 467, 468, 568, 569, 570, 643, 644, and 645 of the nucleic acid as set forth in SEQ ID NO. 1; and/or
   (iib) one or more nucleotide deletion(s) in the sequenced nucleic acid at positions corresponding to positions 205, 206, 207, 208, 209, 210, 430, 431, 432, 469, 470, 471, 472, 473 and 474 of the nucleic acid as set forth in SEQ ID NO. 1; and
(iii) correlating the one or more nucleotide replacement(s) and/or one or more nucleotide deletion(s) with the presence of one or more of SARS-CoV 2 virus omicron, alpha, beta, delta and/or gamma.

5. The method of any one of items 1 to 3, wherein the biological sample is a saliva sample, a nasal sample, a nasopharyngeal sample, a sputum sample, an oropharyngeal sample, a urine sample or a blood sample.

6. The method of any one of items 1 to 3 or 5, wherein the biological sample is a swab sample.

7. The method of any one of items 1 to 3, 5 or 6, wherein the biological sample is a culture supernatant obtained from a culture of a biological sample.

8. The method of any one of items 1 to 3, or 5 to 7, wherein the subject is a human or an animal.

9. The method of any one of items 1 to 3, or 5 to 8, wherein the nucleic acid is comprised in a biological sample that has been obtained from the subject.

10. The method of any one of items 1 to 3, or 5 to 9, wherein the detecting is diagnosing a subject with one or more of SARS-CoV 2 virus omicron, alpha, beta, delta and/or gamma, and wherein the nucleic acid has been derived from a biological sample that has been obtained from the subject.

11. The method of item 2 or 4, wherein the environmental sample is sewage water, or taken from air or surface.

12. The method of any one of items 1 to 11, wherein the SARS-CoV 2 virus omicron comprises nucleotide deletions at positions corresponding to positions 205, 206, 207, 208, 209, and 210 of SEQ ID NO. 1 and one or more nucleotide replacement(s) at positions corresponding to positions 424, 425 and/or 426 of SEQ ID NO.1, and/or wherein the SARS-CoV 2 virus omicron comprises amino acid deletions at positions corresponding to positions 69 and 70 of SEQ ID NO. 2 and an amino acid replacement from G to D at a position corresponding to position 142 of SEQ ID NO.2.

13. The method of any one of items 1 to 12, wherein the SARS-CoV 2 virus alpha comprises nucleotide deletions at positions corresponding to positions 205, 206, 207, 208, 209, 210, 430, 431 and 432 of SEQ ID NO. 1, and/or wherein the SARS-CoV 2 virus alpha comprises amino acid deletions at positions corresponding to positions 69, 70 and 144 of SEQ ID NO. 2.

14. The method of any one of items 1 to 13, wherein the SARS-CoV 2 virus beta comprises one or more nucleotide replacement(s) at positions corresponding to positions 238, 239 and 240 of SEQ ID NO. 1 and one or more nucleotide replacement(s) at positions corresponding to positions 643, 644 and 645 of SEQ ID NO. 1 and/or wherein the SARS-CoV 2 virus beta comprises amino acid replacements from D to A at a position corresponding to position 80 of SEQ ID NO. 2 and from D to G at a position corresponding to position 215 of SEQ ID NO. 2.

15. The method of any one of items 1 to 14, wherein the SARS-CoV 2 virus delta comprises nucleotide deletions at positions corresponding to positions 469, 470, 471, 472, 473, and 474 of SEQ ID NO. 1, one or more nucleotide replacement(s) at positions corresponding to position 424, 425 and/or 426 of SEQ ID NO. 1, and one or more nucleotide replacement(s) at positions corresponding to positions 466, 467 and/or 468 of SEQ ID NO. 1, and/or
wherein the SARS-CoV 2 virus delta comprises amino acid deletions at positions corresponding to positions 157 and 158 of SEQ ID NO. 2, an amino acid replacement from G to D at a position corresponding to position 142 of SEQ ID NO. 2, and an amino acid replacement from E to G at a position corresponding to position 156 of SEQ ID NO. 2.

16. The method of any one of items 1 to 15, wherein the SARS-CoV 2 virus gamma comprises one or more nucleotide replacement(s) at positions corresponding to positions 76, 77 and/or 78 of SEQ ID NO. 1, one or more nucleotide replacement(s) at positions corresponding to position 412, 413 and/or 414 of SEQ ID NO. 1, and one or more nucleotide replacement(s) at positions corresponding to positions 568, 569 and/or 570 of SEQ ID NO.1, and/or wherein the SARS-CoV 2 virus gamma comprises amino acid replacement from P to S at a position corresponding to position 26 of SEQ ID NO. 2, an amino acid replacement from D to Y at a position corresponding to position 138 of SEQ ID NO. 2, and an amino acid replacement from R to S at a position corresponding to position 190 of SEQ ID NO.2.

17. The method of any one of items 1 to 16, wherein the sequencing is performed by Sanger sequencing, Maxam-Gilbert sequencing, shotgun sequencing, Ion Torrent sequencing, pyrosequencing, sequencing by synthesis, Single Molecule Real-Time (SMRT) sequencing or nanopore sequencing.

18. The method of any one of items 1 to 17, wherein the nucleic acid has a length of at most 1400 nucleotides, at most 1300 nucleotides, at most 1200 nucleotides, at most 1100 nucleotides, at most 1000 nucleotides, at most 900 nucleotides, at most 800 nucleotides, at most 700 nucleotides or at most 600 nucleotides or less.

19. The method of any one of items 1 to 18, wherein the method further comprises a step
(a) extracting RNA from a sample.

20. The method of any one of items 1 to 19, wherein the extracting of the RNA is performed using organic extraction, preferably phenol-Guanidine Isothiocyanate (GITC)-based solutions, silica-membrane based spin column technology, or paramagnetic particle technology.

21. The method of any one of items 1 to 20, wherein the method further comprises a step
(b) converting the RNA nucleic acid into the corresponding cDNA nucleic acid.

22. The method of any one of items 1 to 21, wherein the converting of the RNA nucleic acid into the corresponding cDNA nucleic acid is performed by poly-A tagging, or RNase H-DNA polymerase I-mediated second-strand cDNA synthesis.

23. The method of any one of items 1 to 22, wherein the method further comprises step
(c) amplifying the cDNA comprising a cDNA nucleic acid corresponding to positions 76 to 645 of the nucleic acid (cDNA) as set forth in SEQ ID NO. 1, wherein the total amplified cDNA nucleic acid has a length of at most 1500 nucleotides.

24. The method of any one of items 1 to 23, wherein the amplification of the cDNA is performed by PCR.

25. The method of any one of items 1 to 24, wherein the converting of the RNA nucleic acid into the corresponding cDNA nucleic acid and the amplification of the cDNA nucleic acid is performed by RT-PCR.

26. The method of any one of items 1 to 25, wherein the PCR products of the cDNA nucleic acid are purified.

27. The method of any one of items 1 to 26, wherein purification of the cDNA nucleic acid is performed by gel electrophoresis or enzymes.

28. The method of any one of items 1 to 27, wherein the nucleic acid that is sequenced in step (i) is the cDNA nucleic acid obtained in step (c).

29. The method of any one of items 1 to 28, wherein the sequencing of step (i) and/or the amplification of step (c) comprises the use of an oligonucleotide of SEQ ID NO. 28 and/or an oligonucleotide of SEQ ID NO. 29.

30. The method of any one of items 1 to 29, wherein the detecting of the presence or absence of one or more nucleotide replacement(s) and/or one or more nucleotide deletions in the sequenced nucleic acid is performed by comparing the sequenced nucleic acid to the nucleic acid of positions 76 to 645 as set forth in SEQ ID NO. 1 or wherein the method further comprises a step
(e) comparing the sequenced nucleic acid to the nucleic acid sequence of positions 76 to 645 as set forth in SEQ ID NO. 1.

31. The method of any one of items 1 to 30, wherein the method further comprises a step
(d) converting the sequenced nucleic acid into the corresponding amino acid sequence.

32. The method of any one of items 1 to 31, wherein the corresponding amino acid sequence corresponds to positions 26 to 215 of the sequence as set forth in SEQ ID No. 2.

33. The method of any one of items 1 to 32, wherein the detected one or more nucleotide replacement(s) and/or one or more nucleotide deletion(s) in the sequenced nucleic acid correspond to one or more amino acid replacement(s) corresponding to positions 26, 80, 138, 142, 156, 190 and/or 215 and amino acid deletions corresponding to positions 69, 70, 144, 157 and/or 158 of the amino acid sequence set forth in SEQ ID NO. 2.

34. The method of any one of items 1 to 33, wherein the method further comprises step
(f) correlating the one or more nucleotide replacement(s) and/or one or more nucleotide deletion(s) with
   (x) omicron, when nucleotide deletions at positions corresponding to positions 205, 206, 207, 208, 209, and 210 of SEQ ID NO. 1 and one or more nucleotide replacements at positions corresponding to positions 424, 425 and/or 426 of SEQ ID NO.1 are detected;
   (xi) alpha, when nucleotide deletions at positions corresponding to positions 205, 206, 207, 208, 209, 210, 430, 431 and 432 of SEQ ID NO. 1 are detected;
   (xii) beta, when one or more nucleotide replacement(s) at positions corresponding to positions 238, 239 and 240 of SEQ ID NO. 1 and one or more nucleotide replacements at positions corresponding to positions 643, 644 and 645 of SEQ ID NO. 1 are detected;
   (xiii) delta, when nucleotide deletions at positions corresponding to positions 469, 470, 471, 472, 473, and 474 of SEQ ID NO. 1, one or more nucleotide replacements at positions corresponding to position 424, 425 and/or 426 of SEQ ID NO. 1, and one or more nucleotide replacement(s) at positions corresponding to positions 466, 467 and/or 468 of SEQ ID NO. 1 are detected; and/or
   (xiv) gamma, when one or more nucleotide replacement(s) at positions corresponding to positions 76, 77 and/or 78 of SEQ ID NO. 1, one or more nucleotide replacement(s) at positions corresponding to position 412, 413 and/or 414 of SEQ ID NO. 1, and one or more nucleotide replacement(s) at positions corresponding to positions 568, 569 and/or 570 of SEQ ID NO.1 are detected.

35. The method of any one of items 1 to 34, wherein the method further comprises step
(f) correlating the one or more amino acid replacement(s) and/or one or more amino acid deletion(s) with
   (x) omicron, when amino acid deletions at positions corresponding to positions 69 and 70 of SEQ ID NO. 2 and an amino acid replacement from G to D at a position corresponding to position 142 of SEQ ID NO.2 are detected;
   (xi) alpha, when amino acid deletions at positions corresponding to positions 69, 70 and 144 of SEQ ID NO. 2 are detected;
   (xii) beta, when amino acid replacements from D to A at a position corresponding to position 80 of SEQ ID No. 2 and from D to G at a position corresponding to positions 215 of SEQ ID NO. 2 are detected;
   (xiii) delta, when amino acid deletions at positions corresponding to positions 157 and 158 of SEQ ID NO. 2, an amino acid replacement from G to D at a position corresponding to position 142 of SEQ ID NO. 2, and an amino acid replacement from E to G at a position corresponding to position 156 of SEQ ID NO. 2 are detected; and/or
   (xiv) gamma, when an amino acid replacement from P to S at a position corresponding to position 26 of SEQ ID NO. 2, an amino acid replacement from D to Y at a position corresponding to position 138 of SEQ ID NO. 2, and an amino acid replacement from R to S at a position corresponding to position 190 of SEQ ID NO.2 are detected.

36. The method of any one of items 1 to 35, wherein the method further comprises step
(f) diagnosing the subject with SARS-CoV 2 virus omicron, alpha, beta, delta and/or gamma, by correlating the one or more nucleotide replacement(s) and/or one or more nucleotide deletion(s) with
   (x) omicron, when nucleotide deletions at positions corresponding to positions 205, 206, 207, 208, 209, and 210 of SEQ ID NO. 1 and one or more nucleotide replacement(s) at positions corresponding to positions 424, 425 and/or 426 of SEQ ID NO.1 are detected;
   (xi) alpha, when nucleotide deletions at positions and corresponding to positions 205, 206, 207, 208, 209, 210, 430, 431 and 432 of SEQ ID NO. 1 are detected;
   (xii) beta, when one or more nucleotide replacement(s) at positions corresponding to positions 238, 239 and 240 of SEQ ID NO. 1 and one or more nucleotide replacements at positions corresponding to positions 643, 644 and 645 of SEQ ID NO. 1 are detected;
   (xiii) delta, when nucleotide deletions at positions corresponding to positions 469, 470, 471, 472, 473, and 474 of SEQ ID NO. 1, one or more nucleotide replacements at position corresponding to position 424, 425 and/or 426 of SEQ ID NO. 1, and one or more nucleotide replacement(s) at positions corresponding to positions 466, 467 and/or 468 of SEQ ID NO. 1 are detected; and/or
   (xiv) gamma, when one or more nucleotide replacement(s) at positions corresponding to positions 76, 77 and/or 78 of SEQ ID NO. 1, one or more nucleotide replacement(s) at positions corresponding to position 412, 413 and/or 414 of SEQ ID NO. 1, and one or more nucleotide replacement(s) at positions corresponding to positions 568, 569 and/or 570 of SEQ ID NO.1 are detected.

37. The method of any one of items 1 to 36, wherein the method further comprises step
(f) diagnosing the subject with SARS-CoV 2 virus omicron, alpha, beta, delta and/or gamma by correlating the one or more amino acid replacement(s) and/or one or more amino acid deletion(s) with
   (x) omicron, when amino acid deletions at positions corresponding to positions 69 and 70 of SEQ ID NO. 2 and an amino acid replacement from G to D at a position corresponding to position 142 of SEQ ID NO.2 are detected;
   (xi) alpha, when amino acid deletions at positions corresponding to positions 69, 70 and 144 of SEQ ID NO. 2 are detected;
   (xii) beta, when amino acid replacements from D to A at a position corresponding to position 80 of SEQ ID No. 2 and from D to G at a position corresponding to positions 215 of SEQ ID NO. 2 are detected;
   (xiii) delta, when amino acid deletions at positions corresponding to positions 157 and 158 of SEQ ID NO. 2, an amino acid replacement from G to D at a position corresponding to position 142 of SEQ ID NO. 2, and an amino acid replacement from E to G at a position corresponding to position 156 of SEQ ID NO. 2 are detected; and/or
   (xiv) gamma, when an amino acid replacement from P to S at a position corresponding to position 26 of SEQ ID NO. 2, an amino acid replacement from D to Y at a position corresponding to position 138 of SEQ ID NO. 2, and an amino acid replacement from R to S at a position corresponding to position 190 of SEQ ID NO.2 are detected.

38. The method of any one of items 1 to 37, wherein the method comprises
(a) extracting RNA from a sample;
(b) converting the RNA nucleic acid into the corresponding cDNA nucleic acid;
(c) amplifying the cDNA nucleic acid having at least 80% identity to a nucleic acid corresponding to positions 76 to 645 of the nucleic acid as set forth in SEQ ID NO. 1, wherein the amplified cDNA nucleic acid has a length of at most 1500 nucleotides
(i) sequencing the amplified cDNA nucleic acid;
(e) comparing the sequenced cDNA nucleic acid to the sequence of positions 76 to 645 of SEQ ID NO.1;
(ii) identifying from the sequenced cDNA nucleic acid
   (iia) one or more nucleotide replacement(s) at positions corresponding to positions 76, 77, 78, 238, 239, 240, 412, 413, 414, 424, 425, 426, 466, 467, 468, 568, 569, 570, 643, 644, and 645 as set forth in SEQ ID NO. 1; and/or
   (iib) one or more nucleotide deletion(s) in the sequenced nucleic acid at positions corresponding to positions 205, 206, 207, 208, 209, 210, 430, 431, 432, 469, 470, 471, 472, 473, 474 of the nucleic acid encoding the spike protein of the SARS-CoV2 virus as set forth in SEQ ID NO. 1; and
(f) correlating the one or more nucleotide replacement(s) and/or one or more nucleotide deletion(s) with
   (x) omicron, when nucleotide deletions at positions corresponding to positions 205, 206, 207, 208, 209, and 210 of SEQ ID NO. 1 and one or more nucleotide replacement(s) at positions 424, 425 and/or 426 corresponding to positions 424, 425 and/or 426 of SEQ ID NO.1 are detected;
   (xi) alpha, when nucleotide deletions at corresponding to positions 205, 206, 207, 208, 209, 210, 430, 431 and 432 of SEQ ID NO. 1 are detected;
   (xii) beta, when one or more nucleotide replacement(s) at positions corresponding to positions 238, 239 and 240 of SEQ ID NO. 1 and one or more nucleotide replacement(s) at positions corresponding to positions 643, 644 and 645 of SEQ ID NO. 1 are detected;
   (xiii) delta, when nucleotide deletions at positions corresponding to positions 469, 470, 471, 472, 473, and 474 of SEQ ID NO. 1, one or more nucleotide replacements at positions corresponding to position 424, 425 and/or 426 of SEQ ID NO. 1, and one or more nucleotide replacement(s) at positions corresponding to positions 466, 467 and/or 468 of SEQ ID NO. 1 are detected; and/or
   (xiv) gamma, when one or more nucleotide replacement(s) at positions corresponding to positions 76, 77 and/or 78 of SEQ ID NO. 1, one or more nucleotide replacement(s) at positions corresponding to position 412, 413 and/or 414 of SEQ ID NO. 1, and one or more nucleotide replacement(s) at positions 570 corresponding to positions 568, 569 and/or 570 of SEQ ID NO.1 are detected.

39. The method of any one of items 1 to 38, wherein the method comprises step
(e) comparing the sequenced cDNA nucleic acid to a nucleic acid of positions 26 to 215 of SEQ ID NO. 2.

40. The method of any one of items 1 to 39, wherein the method comprises
(a) extracting RNA from the sample;
(b) converting the RNA nucleic acid into the corresponding cDNA nucleic acid;
(c) amplifying the cDNA nucleic acid having at least 80% identity to a nucleic acid corresponding to positions 76 to 645 of the nucleic acid as set forth in SEQ ID NO. 1, wherein the cDNA nucleic acid has a length of at most 1500 nucleotides;
(i) sequencing the amplified cDNA nucleic acid;
(d) converting the sequenced cDNA nucleic acid into the corresponding amino acid sequence;
(e) comparing the amino acid sequence to an amino acid sequence of positions 26 to 215 of SEQ ID NO. 2;
(ii) identifying from the amino acid sequence
   (iia) one or more amino acid replacement(s) at positions corresponding to positions 26, 80, 138, 142, 156, 190 and/or 215 of SEQ ID NO. 2; and/or
   (iib) one or more amino acid deletion(s) at positions corresponding to positions 69, 70, 144, 157 and/or 158 of the protein sequence of the spike protein of the SARS-CoV2 virus as set forth in SEQ ID NO. 2; and
(f) correlating the one or more amino acid replacement(s) and/or one or more amino acid deletion(s) with
   (x) omicron, when amino acid deletions at positions corresponding to positions 69 and 70 of SEQ ID NO. 2 and an amino acid replacement from G to D at a position corresponding to position 142 of SEQ ID NO.2 are detected;
   (xi) alpha, when amino acid deletions at positions corresponding to positions 69, 70 and 144 of SEQ ID NO. 2 are detected;
   (xii) beta, when amino acid replacements from D to A at a position corresponding to position 80 of SEQ ID No. 2 and from D to G at a position corresponding to positions 215 of SEQ ID NO. 2 are detected;
   (xiii) delta, when amino acid deletions at positions corresponding to positions 157 and 158 of SEQ ID NO. 2, an amino acid replacement from G to D at a position corresponding to position 142 of SEQ ID NO. 2, and an amino acid replacement from E to G at a position corresponding to position 156 of SEQ ID NO. 2 are detected; and/or
   (xiv) gamma, when an amino acid replacement from P to S at a position corresponding to position 26 of SEQ ID NO. 2, an amino acid replacement from D to Y at a position corresponding to position 138 of SEQ ID NO. 2, and an amino acid replacement from R to S at a position corresponding to position 190 of SEQ ID NO.2 are detected.

41. The method of any one of items 1 to 40, wherein the PCR products of the cDNA nucleic acid obtained in step (c) and/or the sequenced cDNA products of step (i) are/is purified.

42. The method of any one of item 1 to 41, wherein the method includes a step of correlating the one or more nucleic acid replacement(s) and/or one or more nucleic acid deletion(s) with
(x) omicron, when nucleotide deletions at positions corresponding to positions 205, 206, 207, 208, 209, and 210 of SEQ ID NO. 1 and nucleotide replacement(s)
   from “G” to “A” at a position corresponding to position 425 of SEQ ID NO. 1; or
   from “G” to “A” at a position corresponding to position 425 of SEQ ID NO. 1 and from “T” to “C” at a position corresponding to position 426 of SEQ ID NO. 1 are detected;
(xi) alpha, when nucleotide deletions at positions corresponding to positions 205, 206, 207, 208, 209, 210, 430, 431 and 432 of SEQ ID NO. 1 are detected; (xii) beta, when nucleotide replacement(s):
A)
   from “A” to “C” at a position corresponding to position 239 of SEQ ID NO. 1;
   from “A” to “C” at a position corresponding to position 239 of SEQ ID NO. 1 and from “T to “C” at a position corresponding to position 240 of SEQ ID NO. 1;
   from “A” to “C” at a position corresponding to position 239 of SEQ ID NO. 1 and from “T” to “A” at a position corresponding to position 240 of SEQ ID NO. 1; or
   from “A” to “C” at a position corresponding to position 239 of SEQ ID NO. 1 and from “T” to “G” at a position corresponding to position 240 of SEQ ID NO. 1; and
B)
   from “A” to “G” at a position corresponding to position 644 of SEQ ID NO. 1 and from “T” to “C” at a position corresponding to position 645 of SEQ ID NO. 1;
   from “A” to “G” at a position corresponding to position 644 of SEQ ID NO. 1;
   from “A” to “G” at a position corresponding to position 644 of SEQ ID NO. 1 and from “T” to “A” at a position corresponding to position 645 of SEQ ID NO. 1; or
   from “A” to “G” at a position corresponding to position 644 of SEQ ID NO. 1 and from “T” to “G” at a position corresponding to position 645 of SEQ ID NO. 1 are detected;
(xiii) delta, when nucleotide deletions at positions corresponding to positions 469, 470, 471, 472, 473, and 474 of SEQ ID NO. 1 and nucleotide replacements: A)
   from “G” to “A” at a position corresponding to position 425 of SEQ ID NO. 1; or
   from “G” to “A” at a position corresponding to position 425 of SEQ ID NO. 1 and from “T” to “C” at a position corresponding to position 426 of SEQ ID NO. 1; and
B)
   from “A” to “G” at a position corresponding to position 466 of SEQ ID NO. 1 and from “G” to “C” at a position corresponding to position 467 of SEQ ID NO. 1;
   from “A” to “G” at a position corresponding to position 466 of SEQ ID NO. 1 and from “G” to “T” at a position corresponding to position 467 of SEQ ID NO. 1;
   from “A” to “G” at a position corresponding to position 466 of SEQ ID NO. 1 and from “G” to “A” at a position corresponding to position 467 of SEQ ID NO. 1; or
   from “A” to “G” at a position corresponding to position 466 of SEQ ID NO. 1 are detected; and/or
(xiv) gamma, when nucleotide replacement(s)
A)
   from “C” to “T” at a position corresponding to position 76 of SEQ ID NO. 1;
   from “C” to “T” at a position corresponding to position 76 of SEQ ID NO. 1 and from “T” to “C” at a position corresponding to position 78 of SEQ ID NO. 1;
   from “C” to “T” at a position corresponding to position 76 of SEQ ID NO. 1 and from “T” to “A” at a position corresponding to position 78 of SEQ ID NO. 1;
   from “C” to “T” at a position corresponding to position 76 of SEQ ID NO. 1 and from “T” to “G” at a position corresponding to position 78 of SEQ ID NO. 1;
   from “C” to “A” at a position corresponding to position 76 of SEQ ID NO. 1 and from “C” to “G” at a position corresponding to position 77 of SEQ ID NO. 1; or
   from “C” to “A” at a position corresponding to position 76 of SEQ ID NO. 1, from “C” to “G” at a position corresponding to position 77 of SEQ ID NO. 1 and from “T” to “C” at a position corresponding to position 78 of SEQ ID NO. 1; and
B)
   from “G” to “T” at a position corresponding to position 412 of SEQ ID NO. 1; or
   from “G” to “T” at a position corresponding to position 412 of SEQ ID NO. 1 and from “T” to “C” at a position corresponding to position 414 of SEQ ID NO. 1; and
C)
   from “G” to “C” at a position corresponding to position 570 of SEQ ID NO. 1;
   from “G” to “T” at a position corresponding to position 570 of SEQ ID NO. 1;
   from “A” to “T” at a position corresponding to position 568 of SEQ ID NO. 1, from “G” to “C” at a position corresponding to position 569 of SEQ ID No. 1 and from “G” to “A” at a position corresponding to position 570 of SEQ ID NO. 1;
   from “A” to “T” at a position corresponding to position 568 of SEQ ID No. 1, from “G” to “C” at a position corresponding to position 569 of SEQ ID NO. 1 and from “G” to “T” at a position corresponding to position 570 of SEQ ID NO. 1;
   from “A” to “T” at a position corresponding to position 568 of SEQ ID No. 1, from “G” to “C” at a position corresponding to position 569 of SEQ ID NO. 1 and from “G” to “C” at a position corresponding to position 570 of SEQ ID NO. 1; or
   from “A” to “T” at a position corresponding to position 568 of SEQ ID NO. 1 and from “G” to “C” at a position corresponding to position 569 of SEQ ID NO. 1 are detected.

43. The method of any one of item 1 to 42, wherein the method includes a step of (g) treating the subject diagnosed with SARS-CoV 2 virus omicron, alpha, beta, delta and/or gamma.

44. A kit comprising means for sequencing a nucleic acid comprising a nucleic acid corresponding to positions 76 to 645 of the nucleic acid as set forth in SEQ ID NO. 1; wherein the nucleic acid has a length of at most 1500 nucleotides.

45. The kit of item 44, wherein the means comprise an oligonucleotide of SEQ ID NO. 28 and/or an oligonucleotide of SEQ ID NO. 29.

46. A kit comprising components for preparing a nucleic acid from a sample that has SARS-CoV 2 virus RNA, wherein the nucleic acid has at least 80% identity to a nucleic acid corresponding to positions 76 to 645 of nucleic acid of SEQ ID NO. 1; wherein the nucleic acid has a length of at most 1500 nucleotides.

47. The kit of item 46, wherein the kit comprises one or more, preferably one or two oligonucleotides of SEQ ID NO. 4, 6, 30 31, 9, 11, 13 or 32.

48. The kit of item 46 or 47, wherein the kit comprises one or more, preferably one oligonucleotide of SEQ ID NO. 4, 6, 30 31 and/or one or more, preferably one oligonucleotide of SEQ ID NO. 9, 11, 13 or 32.

49. The kit of any one of items 46 to 48, wherein the kit comprises an oligonucleotide of SEQ ID NO. 28 and/or an oligonucleotide of SEQ ID NO. 29.

50. A composition comprising SARS CoV 2 virus RNA, one or more oligonucleotide primer complementary to the viral RNA and a reverse transcriptase.

51. The composition of item 50, further comprising one or more of dNTPS, one or more DNA polymerase(s), MgCl2, buffer, DTT.

52. The composition of item 50 or 51, wherein the one or more oligonucleotide primer complementary to the viral RNA is an oligonucleotide of any one of SEQ ID. 4, 6, 30 31, 9, 11, 13 or 32.

53. The composition of any one of items 50 to 52, wherein the one or more oligonucleotide primer complementary to the viral RNA is an oligonucleotide is one or more, preferably one oligonucleotide of SEQ ID NO. 4, 6, 30 31 and/or one or more, preferably one oligonucleotide of SEQ ID NO. 9, 11, 13 or 32.

54. The composition of any one of items 50 or 53, wherein the one or more oligonucleotide primer complementary to the viral RNA are the oligonucleotide of SEQ ID 28 and the oligonucleotide of SEQ ID NO. 29.

55. The composition of any one of items 50 to 54, wherein the composition is subjected to sequencing step (i).

56. Preparing a composition as defined in any one of items 50 to 55, which is subjected to sequencing step (i).

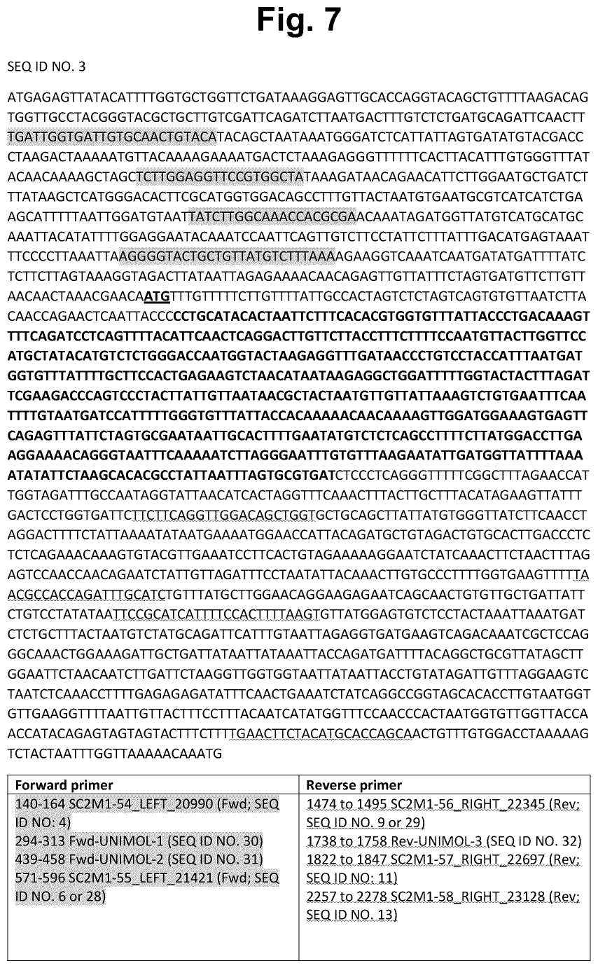

Sequences used in the present invention:

| SEQ | | |
|---|---|---|
| ID | | |
| NO | Description | Sequence |
| 1 | Position | ATGTTTGTTTTTCTTGTTTTATTGCCACTAGTCTCTAGTCAGTGTGTTAATCT |
| | 21563 to | TACAACCAGAACTCAATTACCCCCTGCATACACTAATTCTTTCACACGTGGTG |
| | position | TTTATTACCCTGACAAAGTTTTCAGATCCTCAGTTTTACATTCAACTCAGGAC |
| | 25384 | TTGTTCTTACCTTTCTTTTCCAATGTTACTTGGTTCCATGCTATACATGTCTC |
| | corresponding | TGGGACCAATGGTACTAAGAGGTTTGATAACCCTGTCCTACCATTTAATGATG |
| | to the | GTGTTTATTTTGCTTCCACTGAGAAGTCTAACATAATAAGAGGCTGGATTTTT |
| | spike | GGTACTACTTTAGATTCGAAGACCCAGTCCCTACTTATTGTTAATAACGCTAC |
| | protein of | TAATGTTGTTATTAAAGTCTGTGAATTTCAATTTTGTAATGATCCATTTTTGG |
| | NCBI | GTGTTTATTACCACAAAAACAACAAAAGTTGGATGGAAAGTGAGTTCAGAGTT |
| | identifier | TATTCTAGTGCGAATAATTGCACTTTTGAATATGTCTCTCAGCCTTTTCTTAT |
| | MN908947 | GGACCTTGAAGGAAAACAGGGTAATTTCAAAAATCTTAGGGAATTTGTGTTTA |
| | version 3 | AGAATATTGATGGTTATTTTAAAATATATTCTAAGCACACGCCTATTAATTTA |
| | as of | GTGCGTGATCTCCCTCAGGGTTTTTCGGCTTTAGAACCATTGGTAGATTTGCC |
| | 18 Mar. 2020 | AATAGGTATTAACATCACTAGGTTTCAAACTTTACTTGCTTTACATAGAAGTT |
| | (positions | ATTTGACTCCTGGTGATTCTTCTTCAGGTTGGACAGCTGGTGCTGCAGCTTAT |
| | 76 to 645 | TATGTGGGTTATCTTCAACCTAGGACTTTTCTATTAAAATATAATGAAAATGG |
| | are | AACCATTACAGATGCTGTAGACTGTGCACTTGACCCTCTCTCAGAAACAAAGT |
| | underlined) | GTACGTTGAAATCCTTCACTGTAGAAAAAGGAATCTATCAAACTTCTAACTTT |
| | | AGAGTCCAACCAACAGAATCTATTGTTAGATTTCCTAATATTACAAACTTGTG |
| | | CCCTTTTGGTGAAGTTTTTAACGCCACCAGATTTGCATCTGTTTATGCTTGGA |
| | | ACAGGAAGAGAATCAGCAACTGTGTTGCTGATTATTCTGTCCTATATAATTCC |
| | | GCATCATTTTCCACTTTTAAGTGTTATGGAGTGTCTCCTACTAAATTAAATGA |
| | | TCTCTGCTTTACTAATGTCTATGCAGATTCATTTGTAATTAGAGGTGATGAAG |
| | | TCAGACAAATCGCTCCAGGGCAAACTGGAAAGATTGCTGATTATAATTATAAA |
| | | TTACCAGATGATTTTACAGGCTGCGTTATAGCTTGGAATTCTAACAATCTTGA |
| | | TTCTAAGGTTGGTGGTAATTATAATTACCTGTATAGATTGTTTAGGAAGTCTA |
| | | ATCTCAAACCTTTTGAGAGAGATATTTCAACTGAAATCTATCAGGCCGGTAGC |
| | | ACACCTTGTAATGGTGTTGAAGGTTTTAATTGTTACTTTCCTTTACAATCATA |
| | | TGGTTTCCAACCCACTAATGGTGTTGGTTACCAACCATACAGAGTAGTAGTAC |
| | | TTTCTTTTGAACTTCTACATGCACCAGCAACTGTTTGTGGACCTAAAAAGTCT |
| | | ACTAATTTGGTTAAAAACAAATGTGTCAATTTCAACTTCAATGGTTTAACAGG |
| | | CACAGGTGTTCTTACTGAGTCTAACAAAAAGTTTCTGCCTTTCCAACAATTTG |
| | | GCAGAGACATTGCTGACACTACTGATGCTGTCCGTGATCCACAGACACTTGAG |
| | | ATTCTTGACATTACACCATGTTCTTTTGGTGGTGTCAGTGTTATAACACCAGG |
| | | AACAAATACTTCTAACCAGGTTGCTGTTCTTTATCAGGATGTTAACTGCACAG |
| | | AAGTCCCTGTTGCTATTCATGCAGATCAACTTACTCCTACTTGGCGTGTTTAT |
| | | TCTACAGGTTCTAATGTTTTTCAAACACGTGCAGGCTGTTTAATAGGGGCTGA |
| | | ACATGTCAACAACTCATATGAGTGTGACATACCCATTGGTGCAGGTATATGCG |
| | | CTAGTTATCAGACTCAGACTAATTCTCCTCGGCGGGCACGTAGTGTAGCTAGT |
| | | CAATCCATCATTGCCTACACTATGTCACTTGGTGCAGAAAATTCAGTTGCTTA |
| | | CTCTAATAACTCTATTGCCATACCCACAAATTTTACTATTAGTGTTACCACAG |
| | | AAATTCTACCAGTGTCTATGACCAAGACATCAGTAGATTGTACAATGTACATT |
| | | TGTGGTGATTCAACTGAATGCAGCAATCTTTTGTTGCAATATGGCAGTTTTTG |
| | | TACACAATTAAACCGTGCTTTAACTGGAATAGCTGTTGAACAAGACAAAAACA |
| | | CCCAAGAAGTTTTTGCACAAGTCAAACAAATTTACAAAACACCACCAATTAAA |
| | | GATTTTGGTGGTTTTAATTTTTCACAAATATTACCAGATCCATCAAAACCAAG |
| | | CAAGAGGTCATTTATTGAAGATCTACTTTTCAACAAAGTGACACTTGCAGATG |
| | | CTGGCTTCATCAAACAATATGGTGATTGCCTTGGTGATATTGCTGCTAGAGAC |
| | | CTCATTTGTGCACAAAAGTTTAACGGCCTTACTGTTTTGCCACCTTTGCTCAC |
| | | AGATGAAATGATTGCTCAATACACTTCTGCACTGTTAGCGGGTACAATCACTT |
| | | CTGGTTGGACCTTTGGTGCAGGTGCTGCATTACAAATACCATTTGCTATGCAA |
| | | ATGGCTTATAGGTTTAATGGTATTGGAGTTACACAGAATGTTCTCTATGAGAA |
| | | CCAAAAATTGATTGCCAACCAATTTAATAGTGCTATTGGCAAAATTCAAGACT |
| | | CACTTTCTTCCACAGCAAGTGCACTTGGAAAACTTCAAGATGTGGTCAACCAA |
| | | AATGCACAAGCTTTAAACACGCTTGTTAAACAACTTAGCTCCAATTTTGGTGC |
| | | AATTTCAAGTGTTTTAAATGATATCCTTTCACGTCTTGACAAAGTTGAGGCTG |
| | | AAGTGCAAATTGATAGGTTGATCACAGGCAGACTTCAAAGTTTGCAGACATAT |
| | | GTGACTCAACAATTAATTAGAGCTGCAGAAATCAGAGCTTCTGCTAATCTTGC |
| | | TGCTACTAAAATGTCAGAGTGTGTACTTGGACAATCAAAAAGAGTTGATTTTT |
| | | GTGGAAAGGGCTATCATCTTATGTCCTTCCCTCAGTCAGCACCTCATGGTGTA |
| | | GTCTTCTTGCATGTGACTTATGTCCCTGCACAAGAAAAGAACTTCACAACTGC |
| | | TCCTGCCATTTGTCATGATGGAAAAGCACACTTTCCTCGTGAAGGTGTCTTTG |
| | | TTTCAAATGGCACACACTGGTTTGTAACACAAAGGAATTTTTATGAACCACAA |
| | | ATCATTACTACAGACAACACATTTGTGTCTGGTAACTGTGATGTTGTAATAGG |
| | | AATTGTCAACAACACAGTTTATGATCCTTTGCAACCTGAATTAGACTCATTCA |
| | | AGGAGGAGTTAGATAAATATTTTAAGAATCATACATCACCAGATGTTGATTTA |
| | | GGTGACATCTCTGGCATTAATGCTTCAGTTGTAAACATTCAAAAAGAAATTGA |
| | | CCGCCTCAATGAGGTTGCCAAGAATTTAAATGAATCTCTCATCGATCTCCAAG |
| | | AACTTGGAAAGTATGAGCAGTATATAAAATGGCCATGGTACATTTGGCTAGGT |
| | | TTTATAGCTGGCTTGATTGCCATAGTAATGGTGACAATTATGCTTTGCTGTAT |
| | | GACCAGTTGCTGTAGTTGTCTCAAGGGCTGTTGTTCTTGTGGATCCTGCTGCA |
| | | AATTTGATGAAGACGACTCTGAGCCAGTGCTCAAAGGAGTCAAATTACATTAC |
| | | ACATAA |
| 2 | Spike aa | MFVFLVLLPLVSSQCVNLTTRTQLPPAYTNSFTRGVYYPDKVFRSSVLHSTQD |
| | NCBI | LFLPFFSNVTWFHAIHVSGTNGTKRFDNPVLPFNDGVYFASTEKSNIIRGWIF |
| | identifier | GTTLDSKTQSLLIVNNATNVVIKVCEFQFCNDPFLGVYYHKNNKSWMESEFRV |
| | MN908947 | YSSANNCTFEYVSQPFLMDLEGKQGNFKNLREFVFKNIDGYFKIYSKHTPINL |
| | version 3 | VRDLPQGFSALEPLVDLPIGINITRFQTLLALHRSYLTPGDSSSGWTAGAAAY |
| | as of | YVGYLQPRTFLLKYNENGTITDAVDCALDPLSETKCTLKSFTVEKGIYQTSNF |
| | 18 Mar. 2020 | RVQPTESIVRFPNITNLCPFGEVFNATRFASVYAWNRKRISNCVADYSVLYNS |
| | (positions | ASFSTFKCYGVSPTKLNDLCFTNVYADSFVIRGDEVRQIAPGQTGKIADYNYK |
| | 26 to 215 | LPDDFTGCVIAWNSNNLDSKVGGNYNYLYRLFRKSNLKPFERDISTEIYQAGS |
| | are | TPCNGVEGFNCYFPLQSYGFQPTNGVGYQPYRVVVLSFELLHAPATVCGPKKS |
| | underlined) | TNLVKNKCVNFNFNGLTGTGVLTESNKKFLPFQQFGRDIADTTDAVRDPQTLE |
| | | ILDITPCSFGGVSVITPGTNTSNQVAVLYQDVNCTEVPVAIHADQLTPTWRVY |
| | | STGSNVFQTRAGCLIGAEHVNNSYECDIPIGAGICASYQTQTNSPRRARSVAS |
| | | QSIIAYTMSLGAENSVAYSNNSIAIPTNFTISVTTEILPVSMTKTSVDCTMYI |
| | | CGDSTECSNLLLQYGSFCTQLNRALTGIAVEQDKNTQEVFAQVKQIYKTPPIK |
| | | DFGGFNFSQILPDPSKPSKRSFIEDLLFNKVTLADAGFIKQYGDCLGDIAARD |
| | | LICAQKFNGLTVLPPLLTDEMIAQYTSALLAGTITSGWTFGAGAALQIPFAMQ |
| | | MAYRFNGIGVTQNVLYENQKLIANQFNSAIGKIQDSLSSTASALGKLQDVVNQ |
| | | NAQALNTLVKQLSSNFGAISSVLNDILSRLDKVEAEVQIDRLITGRLQSLQTY |
| | | VTQQLIRAAEIRASANLAATKMSECVLGQSKRVDFCGKGYHLMSFPQSAPHGV |
| | | VFLHVTYVPAQEKNFTTAPAICHDGKAHFPREGVFVSNGTHWFVTQRNFYEPQ |
| | | IITTDNTFVSGNCDVVIGIVNNTVYDPLQPELDSFKEELDKYFKNHTSPDVDL |
| | | GDISGINASVVNIQKEIDRLNEVAKNLNESLIDLQELGKYEQYIKWPWYIWLG |
| | | FIAGLIAIVMVTIMLCCMTSCCSCLKGCCSCGSCCKFDEDDSEPVLKGVKLHY |
| | | T |
| 3 | Position | ATGAGAGTTATACATTTTGGTGCTGGTTCTGATAAAGGAGTTGCACCAGGTAC |
| | 20851 to | AGCTGTTTTAAGACAGTGGTTGCCTACGGGTACGCTGCTTGTCGATTCAGATC |
| | position | TTAATGACTTTGTCTCTGATGCAGATTCAACTTTGATTGGTGATTGTGCAACT |
| | 23175 | GTACATACAGCTAATAAATGGGATCTCATTATTAGTGATATGTACGACCCTAA |
| | corresponding | GACTAAAAATGTTACAAAAGAAAATGACTCTAAAGAGGGTTTTTTCACTTACA |
| | to part | TTTGTGGGTTTATACAACAAAAGCTAGCTCTTGGAGGTTCCGTGGCTATAAAG |
| | of the | ATAACAGAACATTCTTGGAATGCTGATCTTTATAAGCTCATGGGACACTTCGC |
| | spike | ATGGTGGACAGCCTTTGTTACTAATGTGAATGCGTCATCATCTGAAGCATTTT |
| | protein of | TAATTGGATGTAATTATCTTGGCAAACCACGCGAACAAATAGATGGTTATGTC |
| | NCBI | ATGCATGCAAATTACATATTTTGGAGGAATACAAATCCAATTCAGTTGTCTTC |
| | identifier | CTATTCTTTATTTGACATGAGTAAATTTCCCCTTAAATTAAGGGGTACTGCTG |
| | MN908947 | TTATGTCTTTAAAAGAAGGTCAAATCAATGATATGATTTTATCTCTTCTTAGT |
| | version 3 | AAAGGTAGACTTATAATTAGAGAAAACAACAGAGTTGTTATTTCTAGTGATGT |
| | as of | TCTTGTTAACAACTAAACGAACAATGTTTGTTTTTCTTGTTTTATTGCCACTA |
| | 18 Mar. 2020 | GTCTCTAGTCAGTGTGTTAATCTTACAACCAGAACTCAATTACCCCCTGCATA |
| | The start | CACTAATTCTTTCACACGTGGTGTTTATTACCCTGACAAAGTTTTCAGATCCT |
| | codon ATG | CAGTTTTACATTCAACTCAGGACTTGTTCTTACCTTTCTTTTCCAATGTTACT |
| | for the | TGGTTCCATGCTATACATGTCTCTGGGACCAATGGTACTAAGAGGTTTGATAA |
| | spike | CCCTGTCCTACCATTTAATGATGGTGTTTATTTTGCTTCCACTGAGAAGTCTA |
| | protein is | ACATAATAAGAGGCTGGATTTTTGGTACTACTTTAGATTCGAAGACCCAGTCC |
| | underlined | CTACTTATTGTTAATAACGCTACTAATGTTGTTATTAAAGTCTGTGAATTTCA |
| | and | ATTTTGTAATGATCCATTTTTGGGTGTTTATTACCACAAAAACAACAAAAGTT |
| | highlighted | GGATGGAAAGTGAGTTCAGAGTTTATTCTAGTGCGAATAATTGCACTTTTGAA |
| | with bold | TATGTCTCTCAGCCTTTTCTTATGGACCTTGAAGGAAAACAGGGTAATTTCAA |
| | letters, | AAATCTTAGGGAATTTGTGTTTAAGAATATTGATGGTTATTTTAAAATATATT |
| | position 76 | CTAAGCACACGCCTATTAATTTAGTGCGTGATCTCCCTCAGGGTTTTTCGGCT |
| | to 645 are | TTAGAACCATTGGTAGATTTGCCAATAGGTATTAACATCACTAGGTTTCAAAC |
| | highlighted | TTTACTTGCTTTACATAGAAGTTATTTGACTCCTGGTGATTCTTCTTCAGGTT |
| | with bold | GGACAGCTGGTGCTGCAGCTTATTATGTGGGTTATCTTCAACCTAGGACTTTT |
| | letters; | CTATTAAAATATAATGAAAATGGAACCATTACAGATGCTGTAGACTGTGCACT |
| | see also | TGACCCTCTCTCAGAAACAAAGTGTACGTTGAAATCCTTCACTGTAGAAAAAG |
| | FIG. 7 | GAATCTATCAAACTTCTAACTTTAGAGTCCAACCAACAGAATCTATTGTTAGA |
| | | TTTCCTAATATTACAAACTTGTGCCCTTTTGGTGAAGTTTTTAACGCCACCAG |
| | | ATTTGCATCTGTTTATGCTTGGAACAGGAAGAGAATCAGCAACTGTGTTGCTG |
| | | ATTATTCTGTCCTATATAATTCCGCATCATTTTCCACTTTTAAGTGTTATGGA |
| | | GTGTCTCCTACTAAATTAAATGATCTCTGCTTTACTAATGTCTATGCAGATTC |
| | | ATTTGTAATTAGAGGTGATGAAGTCAGACAAATCGCTCCAGGGCAAACTGGAA |
| | | AGATTGCTGATTATAATTATAAATTACCAGATGATTTTACAGGCTGCGTTATA |
| | | GCTTGGAATTCTAACAATCTTGATTCTAAGGTTGGTGGTAATTATAATTACCT |
| | | GTATAGATTGTTTAGGAAGTCTAATCTCAAACCTTTTGAGAGAGATATTTCAA |
| | | CTGAAATCTATCAGGCCGGTAGCACACCTTGTAATGGTGTTGAAGGTTTTAAT |
| | | TGTTACTTTCCTTTACAATCATATGGTTTCCAACCCACTAATGGTGTTGGTTA |
| | | CCAACCATACAGAGTAGTAGTACTTTCTTTTGAACTTCTACATGCACCAGCAA |
| | | CTGTTTGTGGACCTAAAAAGTCTACTAATTTGGTTAAAAACAAATG |

### EXAMPLES

### Example 1. Concept of the Technique

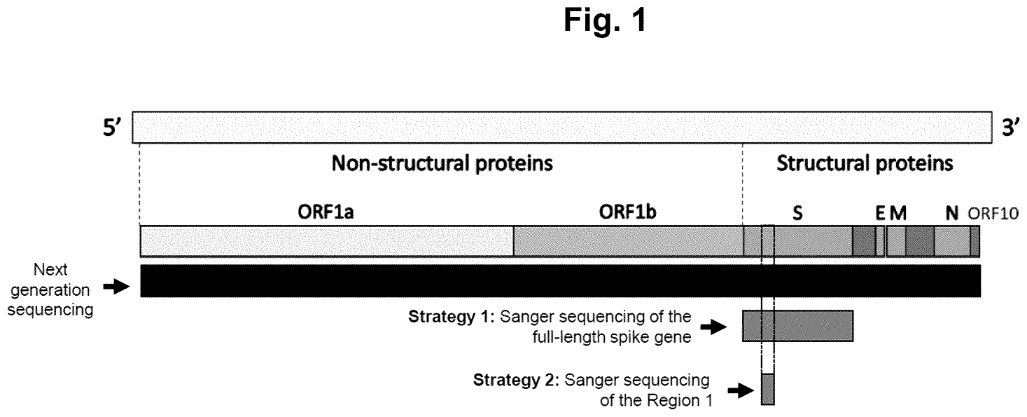

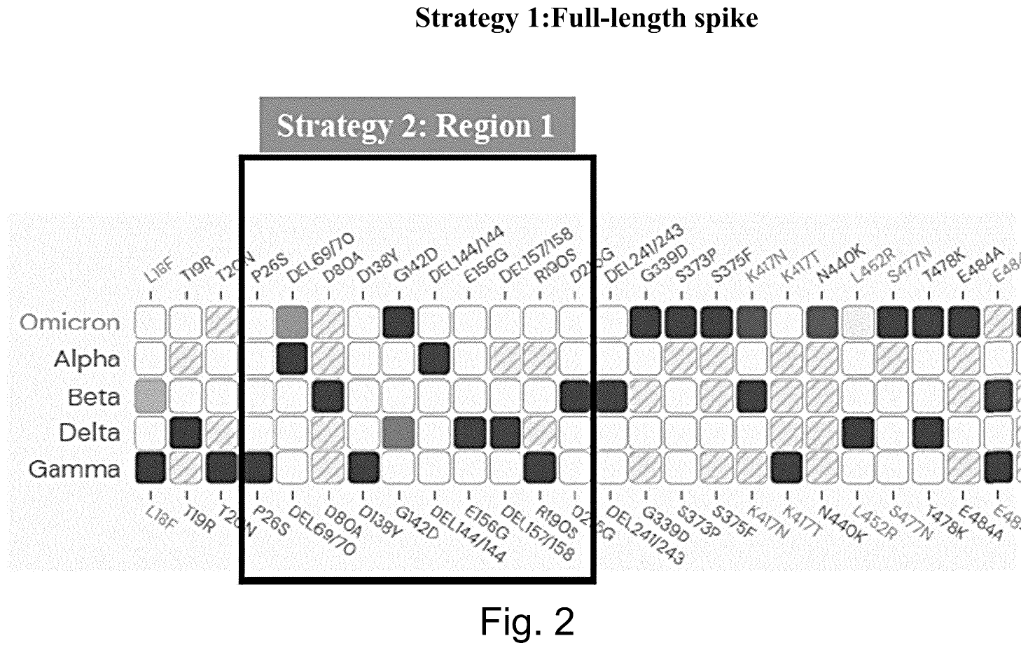

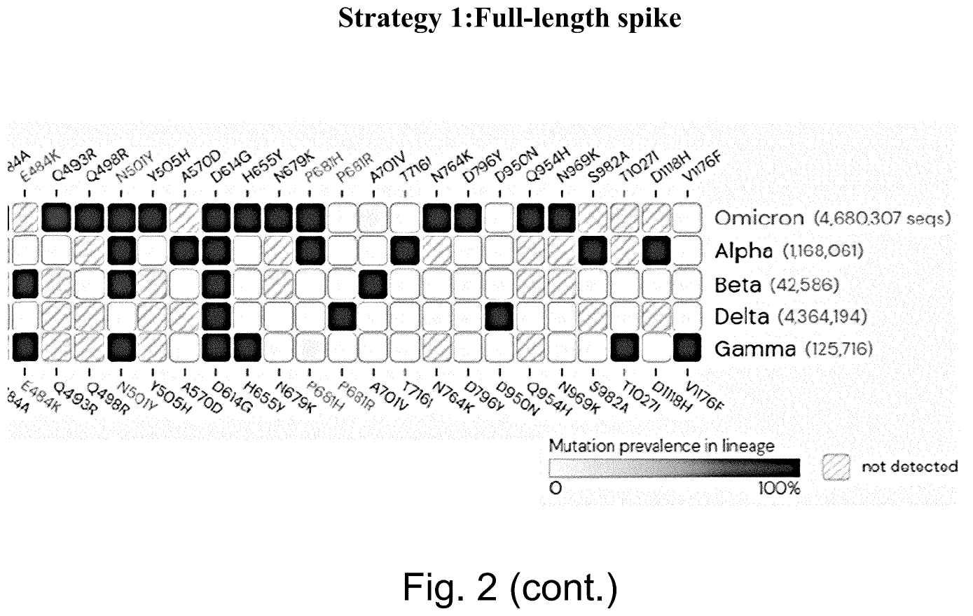

The concept of the technique was initially developed in silico to obtain two Sanger sequencing strategies (FIG. 1), which primarily identify variants of SARS-CoV-2 concern (FIG. 2) (webpage Tracking SARS-CoV-2 variants).

For the first strategy, a total of 12 pairs of oligonucleotides hybridizing to overlapping regions of the viral spike gene were used, which are amplified and sequenced in individual reactions (FIGS. 1 and 2). On the other hand, two other pairs of oligonucleotides are used to amplify and sequence of a specific region of the spike gene; Region 1 (FIGS. 1 and 2). Thus, strategy 2 allows the characterization of gene Region 1 (FIGS. 1 and 2). It is important to mention that each sequencing strategy was designed and developed to be executed independently, and without depending on the previous results of one of them for the issuance of final results.

The second sequencing strategy was chosen because this is a region in which mutations characteristic of each SARS-CoV-2 variant of concern is found (FIG. 2). For example, for sequencing strategy 2 (Region 1), “DEL69/70” is found in 56.4% of Omicron, 96.6% of Alpha, 0.1% of Beta, 0.2% of Delta and Gamma variants. In that sense, detection of the “DEL96/70” mutation allows discriminating with very high probability and reliability Omicron and Alpha from other variants of concern. However, the detection of the “G142D” mutation would allow discriminating Omicron from Alpha since, in Omicron this mutation is present in 95.9% of the sequences described, while in Alpha it has been described in <0.1%. Table 1 summarizes the characteristic mutations that are detected by the second sequencing strategy according to each SARS-CoV-2 variant of concern.

TABLE 1
Characteristic mutations of each SARS-CoV-2 variant of concern
according to sequencing strategy. Detection of the mutations listed
allows for reliable and rapid in silico characterization.

| Variant of concern | Segunda estrategia de secuenciación - Región 1 |
|---|---|
| Omicron | DEL69/70 + G142D |
| Alpha | DEL69/70 + DEL144/144 |
| Beta | D80A + D215G |
| Delta | G142D + E156G + DEL157/158 |
| Gamma | P26S + D138Y + R190S |

### Example 2. Oligonucleotides Used in the Technique

The sequences were obtained from a rapid and sensitive strategy based on the use of six pools of oligonucleotides that allow sequencing of the complete SARS-CoV-2 genome (Paden et al. 2020). The strategy published by Paden et al. 2020 thus allows sequencing of the genome using amplicons of an average size of 500 base pairs that comprise overlapping regions. The sequencing strategy described by these researchers has made it possible to characterize complete genomes using Sanger and other sequencing technologies.

The oligonucleotides used in the strategy described by Paden et al 2020 were aligned to the spike-in gene (Reference: Wuhan-Hu-112019; GenSank accession number: MN908947) to identify hybridization sites and regions of coverage. From those alignments, specific oligonucleotide sets were chosen for the strategies described in this paper. Table 2 lists the oligonucleotides used according to each sequencing strategy and the size of the PCR product according to oligonucleotide combination.

TABLE 2
List of oligonucleotides
used by sequencing strategy.

| | | | Number of |
|---|---|---|---|
| | | | basepairs |
| SEQ | | | of PCR |
| ID | Name | Secuencia (5′-3′) | product |
| | First strategy | | |
| 4 | SC2M1- | TGATTGGTGATTGTGCA | 572 |
| | 54_LEFT_20990 | ACTGTACA | |
| 5 | SC2M1- | TGTTCGTTTAGTTGTTA | |
| | 54_RIGHT_21562 | ACAAGAACATCA | |
| 6 | SC2M1- | AGGGGTACTGCTGTTAT | 495 |
| | 55_LEFT_21421 | GTCTTTAAA | |
| 7 | SC2M1- | AAGTAGGGACTGGGTCT | |
| | 55_RIGHT_21916 | TCGAA | |
| 8 | SC2M1- | TGGGACCAATGGTACTA | 570 |
| | 56_LEFT_21775 | AGAGGT | |
| 9 | SC2M1- | ACCAGCTGTCCAACCTG | |
| | 56_RIGHT_22345 | AAGAA | |
| 10 | SC2M1- | GTGATCTCCCTCAGGGT | 494 |
| | 57_LEFT_22203 | TTTTCG | |
| 11 | SC2M1- | ACTTAAAAGTGGAAAAT | |
| | 57_RIGHT_22697 | GATGCGGAA | |
| 12 | SC2M1- | ACTTGTGCCCTTTTGGT | 565 |
| | 58_LEFT_22563 | GAAGT | |
| 13 | SC2M1- | TGCTGGTGCATGTAGAA | |
| | 58_RIGHT_23128 | GTTCA | |
| 14 | SC2M1- | CCGGTAGCACACCTTGT | 533 |
| | 59_LEFT_22986 | AATGG | |
| 15 | SC2M1- | CCCCTATTAAACAGCCT | |
| | 59_RIGHT_23519 | GCACG | |
| 16 | SC2M1- | ACCAGGTTGCTGTTCTT | 497 |
| | 60_LEFT_23379 | TATCAGG | |
| 17 | SC2M1- | CAGCTATTCCAGTTAAA | |
| | 60_RIGHT_23876 | GCACGGT | |
| 18 | SC2M1- | AATTCTACCAGTGTCTA | 494 |
| | 61_LEFT_23737 | TGACCAAGAC | |
| 19 | SC2M1- | GCACCAAAGGTCCAACC | |
| | 61_RIGHT_24231 | AGAAG | |
| 20 | SC2M1- | GCTGCTAGAGACCTCAT | 528 |
| | 62_LEFT_24095 | TTGTGC | |
| 21 | SC2M1- | AAGCTCTGATTTCTGCA | |
| | 62_RIGHT_24623 | GCTCT | |
| 22 | SC2M1- | AAATGATATCCTTTCAC | 510 |
| | 63_LEFT_24493 | GTCTTGACAAA | |
| 23 | SC2M1- | TGAGTCTAATTCAGGTT | |
| | 63_RIGHT_25003 | GCAAAGGA | |
| 24 | SC2M1- | GCACACACTGGTTTGTA | 511 |
| | 64_LEFT_24858 | ACACAA | |
| 25 | SC2M1- | TTTGACTCCTTTGAGCA | |
| | 64_RIGHT_25369 | CTGGC | |
| 26 | SC2M1- | CTAGGTTTTATAGCTGG | 576 |
| | 65_LEFT_25214 | CTTGATTGC | |
| 27 | SC2M1- | CATTTCCAGCAAAGCCA | |
| | 65_RIGHT_25790 | AAGCC | |
| | Second strategy | | |
| 28 | SC2M1- | AGGGGTACTGCTGTTAT | 924 |
| | 55_LEFT_21421 | GTCTTTAAA | |
| 29 | SC2M1- | ACCAGCTGTCCAACCTG | |
| | 56_RIGHT_22345 | AAGAA | |
| | Further primers that may | | |
| | be alternatively or additionally | | |
| | used in second strategy | | |
| 30 | Fwd-UNIMOL-1 | TCTTGGAGGTTCCGTGG | |
| | | CTA | |
| 31 | Fwd-UNIMOL-2 | TATCTTGGCAAACCACG | |
| | | CGA | |
| 4 | SC2M1- | TGATTGGTGATTGTGCA | |
| | 54_LEFT_20990 | ACTGTACA | |
| 32 | Rev-UNIMOL-3 | GATGCAAATCTGGTGGC | |
| | | GTTA | |
| 11 | SC2M1- | ACTTAAAAGTGGAAAAT | |
| | 57_RIGHT_22697 | GATGCGGAA | |
| 13 | SC2M1- | TGCTGGTGCATGTAGAA | |
| | 58_RIGHT_23128 | GTTCA | |

Overall, it was verified that the characteristic mutations of each SARS-CoV-2 variant of concern (website: //outbreak.info/compare-lineages) are not found in regions where the oligonucleotides align. The oligonucleotides used in the strategies have been previously published (Paden et al. 2020), and used under conditions different from those described in this paper. Specifically, the changes proposed in this paper include modifications in the RT-PCR reaction volumnes, and changes in the cycling conditions and number of cycles in the PCR.

For the first strategy, since 12 pairs of oligonucleotides are used, it was subsequently necessary to perform a reference assembly (Reference: Wuhan-Hu-1/2019; GenBank accession number: MN908947) with the 12 fragments. Similarly, for the second sequencing strategy it was necessary to perform the same assembly using the same genetic reference.

### Example 3. Checking the Validity of the Oligonucleotides

All oligonucleotides were aligned with the first 20 references of the described SARS-CoV-2 variants of concern (webpage Tracking SARS-CoV-2 variants) circulating in Colombia and using information deposited in GISAID (website: //www.gisaid.org/). Specifically, the first 20 sequences of each variant of concern and another 20 sequences of the Mu variant of interest were downloaded, and the oligonucleotides used in each strategy were aligned using MEGA (Kumar et al. 2018).

In silico results demonstrated that all oligonucleotides used in the sequencing strategies allow detection of the SARS-CoV-2 variants of concern and the Mu variant. After this verification, we proceeded with the standardization of the RT-PCR amplification technique and subsequent sequencing of the PCR products.

### Example 4. Standardization of the Technique

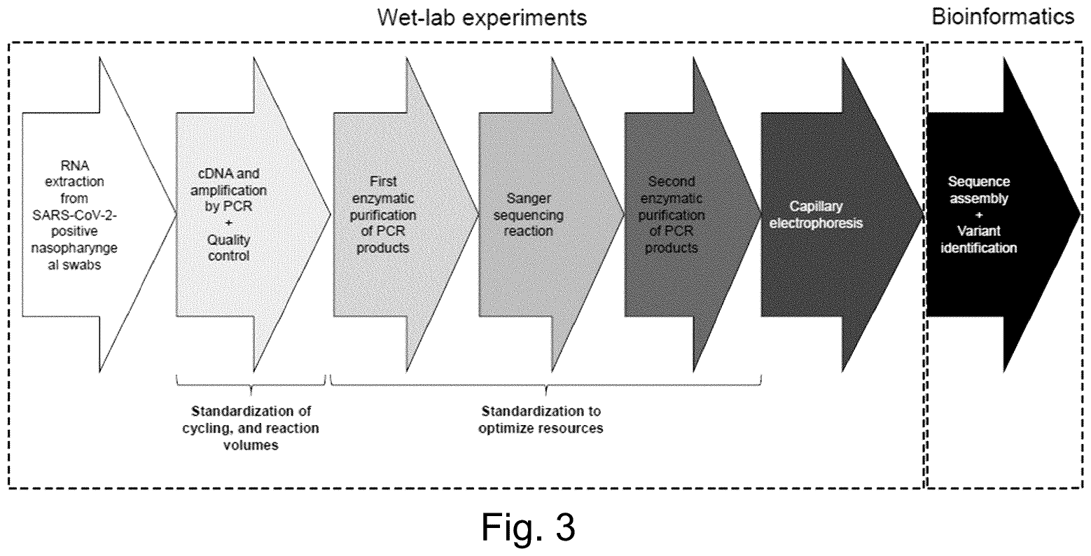

Standardization of the RT-PCR technique (FIG. 2) was aimed at determining the appropriate cycling conditions and reagent volumes in order to amplify the complete ORF of the spike gene in 12 independent reactions and under identical conditions (Strategy 1, FIG. 1). Region 1 (Strategy 2, FIG. 1) were also amplified in independent reactions and under the same conditions. Similarly, multiple controls and steps were included to evaluate the quality of the process and the generation of amplification products. Subsequently, changes in the reaction volumes of the commercial reagents used for amplification were evaluated using dideoxynucleotides and subsequent Sanger sequencing. FIG. 3 shows all the steps to be followed for each sequencing strategy described in this document.

### Example 5. Utilizing the Techniques

RNA extraction was performed with the kit “QIAamp™ Viral RNA Mini Kit” (Manufacturer; Qiagen, Catalog number: 52906) and following the manufacturer's instructions. However, the use of kits that ensure the extraction of high quality RNA at adequate concentrations is recommended.

For the formation of cDNA and subsequent generation of PCR products, the kit “SuperScript™ μl One-Step RT-PCR System with Platinum Taq DNA Polymerase” (Manufacturer: Invitrogen; Catalog number: 12574026) was used. The use of this kit made it possible to work in a single step and in a single 20 μL reaction. Thus, the first sequencing strategy consisted of 12 reactions, and the second and third sequencing strategies consisted of one reaction each. Each RT-PCR reaction consisted of; 10 μL of 2× buffer, 3.6 μL of MgSO4 (5 mM), 0.4 μL of enzyme mix, 1 μL of oligonucleotide pool (Forward 10 mM+Reverse 10 mM), and 5 μL of extracted RNA. The RT-PCR reaction was run on a ProFlex™ thermal cycler (Manufacturer: Applied Biosystems; Catalog number: 4484073) and following the following conditions; 50° C. for 15 min, 94° C. for 2 min, followed by a total of 28 cycles at 94° C. for 30 sec, 60° C. for 1 min 30 sec, and 72° C. for 1 min 30 sec, and finished by one cycle at 72° C. for 10 min and one at 4° C. for indefinite time.

In order to establish a quality control point, an agarose gel electrophoresis step was included. This checkpoint was designed to verify the amplification of each segment. A 1 5% agarose gel was prepared using “UltraPure agarose” (Manufacturer: Invitrogen; Catalog number: 16500100) for visualization of the amplicons. Electrophoresis was run at 110 volts for 30 min on Owl EasyCast™ B1A Mini Gel support (Manufacturer: Thermo Scientific; Catalog number: B1A-UVT) and using a 100 base pair molecular weight marker (Manufacturer: Invitrogen; Catalog number: 15623050). Overall, all sequencing strategies generated PCR products according to the expected size (Table 1).

The first enzymatic purification of PCR products was performed using a kit. According to the experiments performed at UNIMOL, we observed no significant differences in sequencing quality when using the “ExoSAP-IT™ PCR Product Cleanup Reagent” kit (Manufacturer: Applied Biosystems; Catalog number: 78200.200.UL) or the “ExoSAP-IT™ Express PCR Product Cleanup Reagent” kit (Manufacturer: Applied Biosystems; Catalog number: 75001.200.UL). Therefore, this purification step can be performed with any of the previously mentioned kits. The purification steps were performed on a ProFlexM thermal cycler (Manufacturer: Applied Biosystems; Catalog number: 4484073) as described by the manufacturer using 5 μL of PCR product and 2 μL of purification reagent.

After purification of the PCR products, sequencing amplification was performed using the “BigDye Terminator™ v3.1 Cycle Sequencing” kit (Manufacturer: Applied Biosystems; Catalog number: 4337455). It is important to mention that for each PCR reaction two reactions were prepared; one with the Forward and one with the Reverse oligonucleotide. The reaction volumes were modified and standardized to; 4 μL of BigDye Terminator™ reagent, 4 μL of nuclease-free water, 1 μL of oligonucleotide (3.2 μM), and 1 μL of purified PCR product. The volumes were adjusted to optimize reagent utilization. The reaction was run on a ProFlex™ thermal cycler (Manufacturer: Applied Biosystems; Catalog number: 4484073) considering the following conditions; 1 cycle at 96° C. for 1 min, followed by 25 cycles of 96° C. for 10 sec, 50° C. for 5 sec, and 60° C. for 4 min. A final cycle at 4° C. was added for indefinite time.

The second enzymatic purification of PCR products was performed with the “BigDye XTerminatori™ Purification” kit (Manufacturer: Applied Biosystems; Catalog number: 4376486) and following the manufacturer's instructions. Specifically, the purification was run using 45 μL of the SAM solution and with 10 μL of the product from the previous step. The mixture was continuously rotated at 2000 rpm for 30 min in the Basic Vortex Mixer (Manufacturer: Thermo Scientific; Catalog number: 88882012), After mixing, the reaction tubes were centrifuged for 2 min to bring the contents to the bottom of the tube.

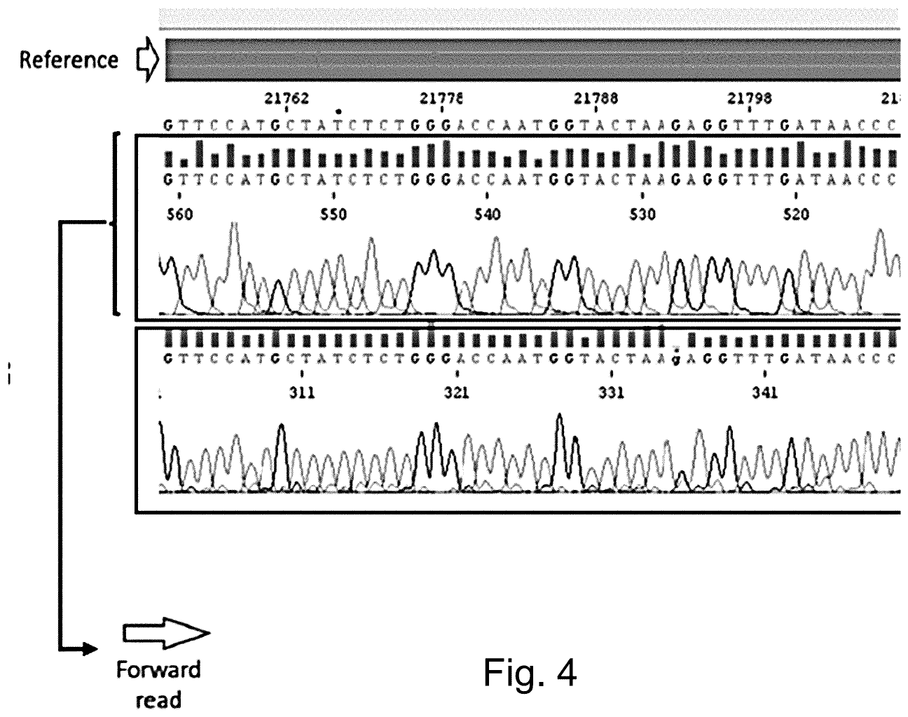

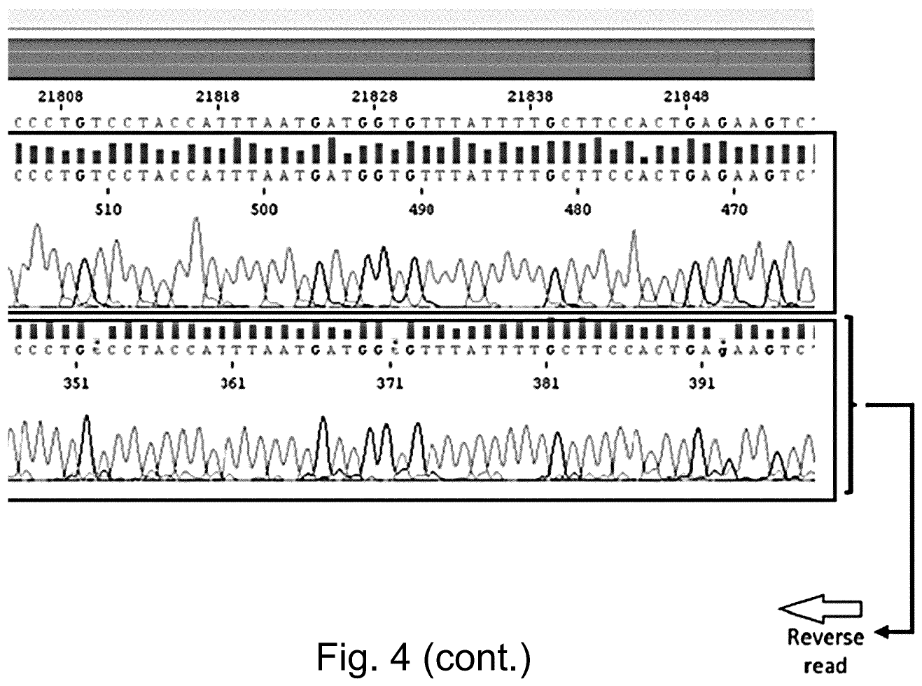

Capillary electrophoresis was run on the SeqStudio™ Genetic Analyzer (Manufacturer: Applied Biosystems; Catalog number: A35644) using the “Sequencing” application, the dye set “Z_BigDye Terminator™ v3.1”, and the “Longseq™_BDX” run module. It is important to mention that the information generated was stored in files with extension “AB1”, and since, two sequencing reactions per PCR product were used, two “AB1” files were finally generated per sequenced product (FIG. 4).

Bioinformatics analysis and quality control of the generated information was analyzed with Sequencing Analysis Software v6.0 (Manufacturer: Applied Biosystems; Catalog number: 4474950), and high quality sequences were mapped by reference in SeqScapeT Software v3.0 (Manufacturer: Applied Biosystems; Catalog number: 4474978). After manually curating the sequences using information obtained for the Forward and Reverse reads (FIG. 4), files with extension “fasta” were generated which formed the final consensus of both reads for each PCR product.

### Example 6. Sequencing Strategy Validation

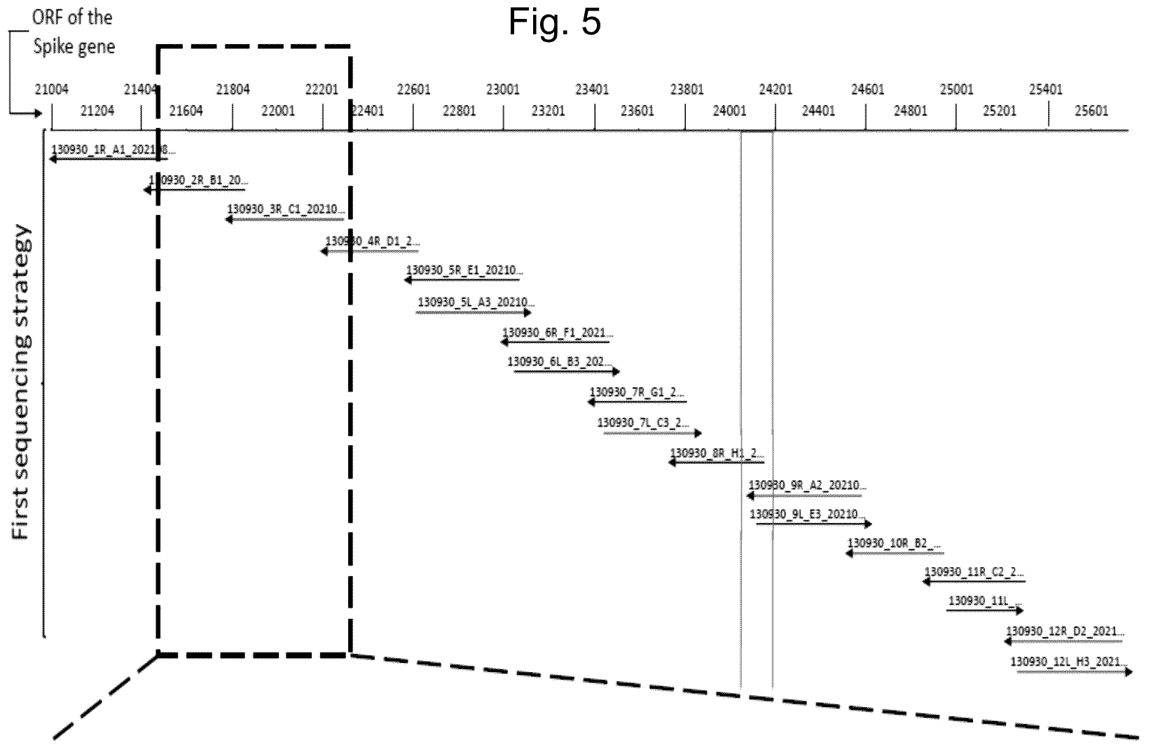

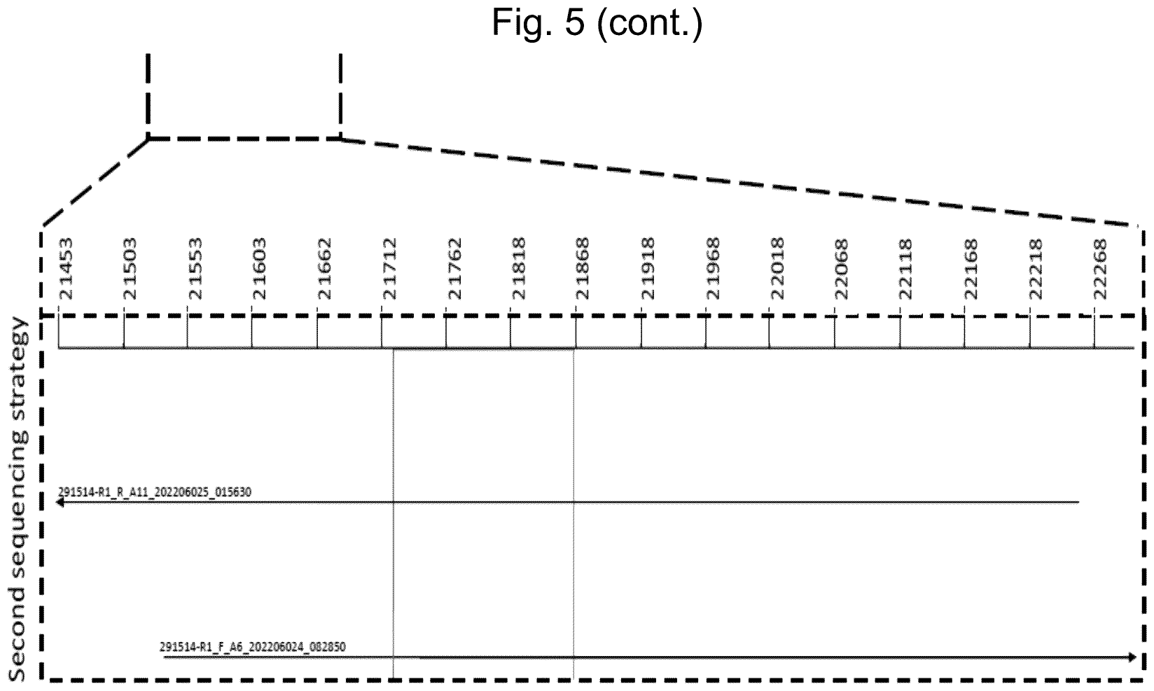

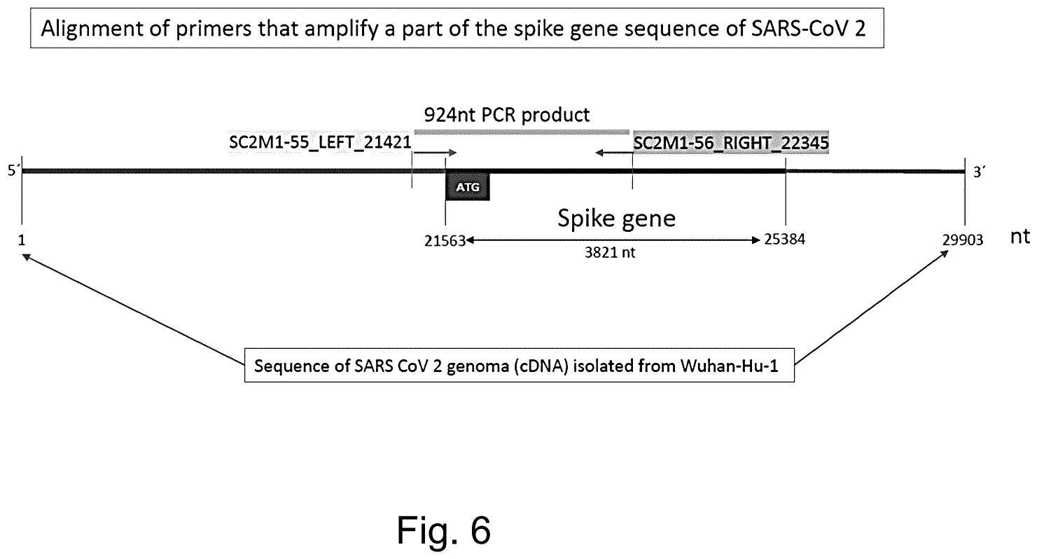

In a first stage, the validation consisted of evaluating the discriminatory power of SARS-CoV-2 variants in a small group of samples (n=10) using the three sequencing strategies (FIGS. 1 and 2). The results of this first stage showed that the first and second sequencing strategies were able to identify and discriminate SARS-CoV-2 variants in the same way and with the same level of accuracy (Table 3 and FIG. 5). It is important to mention that for validation purposes only, the three sequencing strategies were run simultaneously on the reduced set of samples (n=10), However, the execution of each strategy is independent and does not require parallel runs or the need to apply one strategy followed by another. Table 3 summarizes the system of application of each strategy and summarizes the findings for the first stage of validation.

TABLE 3
Characteristics, results and application system of each
sequencing strategy during the first validation stage.

| | Strategy 1 | |
|---|---|---|
| | (complete | Strategy 2 |
| | gene) | Region 1 |
| Number of amplified segments | 12 | 1 |
| Number of sequencing reactions | 24 | 2 |
| Total sequencing time | 6X | 1X |
| Total cost related to sequencing | 6X | 1X |
| SARS-CoV-2 can be detected with the data | yes | yes |
| generated. | | |
| Clade assignment is possible with the generated | yes | yes |
| data | | |
| Requires the execution of other Sanger | No | No |
| sequencing strategies for correct clade | | |
| assignment. | | |
| Conclusive results | yes | yes |
| Report results | yes | yes |

In the second validation step, the sample size was expanded to a total of 37 SARS-CoV-2 variants of concern and 11 SARS-CoV-2 Mu variants. The latter variant was included in the analysis since it is a variant that presumably emerged in Colombia (Laiton-Donato et al. 2021). Thus, a total of 102 SARS-CoV-2 variants were included and sequenced by the next-generation sequencing strategy and by the second Sanger sequencing strategy. The validation of the second sequencing strategy was performed using as reference information generated by a new generation sequencing technique that allows sequencing complete genomes using a standard working protocol designed to work with the MinION (website: //www.protocols.io/view/ncov-2019-sequencing-protocol-v3-locost-bp2l6n26rgqe/v3).

Specifically, this second stage of validation included Gamma (n=2), Delta (n=12), Omicron (n=73) and Mu (n=11) variants. Alpha and Beta variants were not included since they were not detected in UNIMOL, and therefore access to both variants was not possible. Overall, the second Sanger sequencing strategy, which was developed, standardized and validated at UNIMOL, correlated perfectly with clade assignment (website: //clades.nextstrain.org/) with a Kappa index=1.000), and had a percentage of similarity at the nucleotide level in the range of 97.6% to 100.0% compared to the information generated by the next generation sequencing technique.

In summary, the discriminative power of the second sequencing strategy was optimal and could be considered as a rapid screening technique to identify SARS-COV-2 variants. Although Alpha and Beta variants were not included in a formal evaluation, in silico analyses suggest that the discriminatory power would remain equally high and adequate. It is also important to mention that the execution of the second strategy alone provides highly reliable and trustworthy results and does not require the simultaneous and subsequent execution of the first or third sequencing strategy.

Table 4 summarizes multiple technical aspects related to genetic coverage, discriminatory power of SARS-CoV-2 variants, processing capacities and times, approximate costs, and requirements for computational capacity, personnel and training level. It is important to mention that the aspects compared could vary according to future optimizations of the reference technique.

TABLE 4
Comparison of the new generation sequencing technique
used as reference and the Sanger sequencing technique.

| | Sequencing | |
|---|---|---|
| | new | Sequencing |
| Compared characteristics | generation (^{a}) | Sanger |
| Genetic coverage | complete | strategy 2; |
| | gene | region 1 |
| Detects and identifies variants, and allows lineage | yes | yes |
| assignment | | |
| Minimum/maximum sample capacity per process | 24-72 | 1-48 |
| Average time of work in the laboratory per process | 28 | 10 |
| (hours) | | |
| Sequencing time on equipment for minimum and | 12-60 | 4-48 |
| maximum capacity (hours) | | |
| Approximate time spent for analysis of results for | 4-24 | 0.5-12 |
| minimum and maximum capacity (hours) | | |
| Approximate total time for maximum process capacity; | 192 | 108 |
| from RT-PCR to bioinformatics analysis (hours) | | |
| Approximate total time for minimum process capability; | 140 | 40 |
| from RT-PCR to bioinformatics analysis (hours) | | |
| Estimated reference cost (reagents) | 18 X | 1 X |
| Computational requirement for data analysis | Very high | Low+ |
| Laboratory personnel/level of training | 2/high | 1/Medium |

### PUBLICATION LIST

World Health Organization. Coronavirus (COVID-19) events as they happen. [Internet]. [cited 2022 Mar. 10]. Available from: website: //www.who.int/emergencies/diseases/novel-coronavirus-2019/events-as-they-happen
Pekar J, Worobey M, Moshiri N, Scheffler K, Wertheim J O. Timing the SARS-CoV-2 index case in Hubei province. Science. 2021 April; 372(6540):412-7.
COVID Live—Coronavirus Statistics—Worldometer.
SARS-CoV-2 Variant Classifications and Definitions [Internet]. [cited 2022 Mar. 30]. Available from: website: //www.cdc.gov/coronavirus/2019-ncov/variants/variant-classifications.html
Tracking SARS-CoV-2 variants [Internet]. [cited 2022 Mar. 30]. Available from: website: //www.who.int/en/activities/tracking-SARS-CoV-2-variants/
Meredith L W, Hamilton W L, Warne B, Houldcroft C J, Hosmillo M, Jahun A S, et al. Rapid implementation of SARS-CoV-2 sequencing to investigate cases of health-care associated COVID-19: a prospective genomic surveillance study. Lancet Infect Dis. 2020 November; 20(11):1263-71.
Laiton-Donato K, Franco-Munoz C, Alvarez-Diaz D A, Ruiz-Moreno H A, Usme-Ciro J A, Prada D A, et al. Characterization of the emerging B.1.621 variant of interest of SARS-CoV-2. Infect Genet Evol J Mol Epidemiol Evol Genet Infect Dis. 2021 November; 95:105038.
What is Genomic Surveillance? I CDC [Internet]. [cited 2022 Mar. 30]. Available from: website: //www.cdc.gov/coronavirus/2019-ncov/variants/genomic-surveillance.html
Riemersma K K, Grogan B E, Kita-Yarbro A, Halfmann P, Kocharian A, Florek K R, et al. Shedding of Infectious SARS-CoV-2 Despite Vaccination, medRxiv. 2021;
Stoddard G, Black A, Ayscue P, Lu D, Kamm J, Bhatt K, et al. Using genomic epidemiology of SARS-CoV-2 to support contact tracing and public health surveillance in rural Humboldt County, California, medRxiv. 2021;
Chen Z, Azman A S, Chen X, Zou J, Tian Y, Sun R, et al. Global landscape of SARS-CoV-2 genomic surveillance and data sharing. Nat Genet. 2022;
Paden C R, Tao Y, Queen K, Zhang J, Li Y, Uehara A, et al. Rapid, Sensitive, Full-Genome Sequencing of Severe Acute Respiratory Syndrome Coronavirus 2. Emerg Infect Dis. 2020 October; 26(10):2401-5;
Kumar S, Stecher G, Li M, Knyaz C, Tamura K. MEGA X: Molecular Evolutionary Genetics Analysis across Computing Platforms. Mol Biol Evol. 2018/05/04. 2018; 35(6):1547-9
Matranga et al. (2016) Unbiased Deep Sequencing of RNA Viruses from Clinical Samples” Journal of Visualized Experiments, 113, e54117, pp. 1-9
Deng et al. (2005)“Comparison of six RNA extraction methods for the detection of classical swine fever virus by real-time and conventional reverse transcription-PCR” J Vet Diagn Invest 17:574-578
Wozniak et al. (2020) “A simple RNA preparation method for SARS-CoV-2 detection by RT-qPCR” Scientific Reports:16608
Ozsolak and Milos (2011) “RNA sequencing: advances, challenges and opportunities” Nat Rev Genet. 2011 February; 12(2): 87-98
Klerk et al. (2014) “RNA sequencing: from tag-based profiling to resolving complete transcript structure” Cell. Mol. Life Sci. (2014) 71:3537-3551
Sanger et al. (1977) “DNA sequencing with chain-terminating inhibitors” Proc. Nati. Acad. Sci. USA Vol. 74, No. 12, pp. 5463-5467
Altschul. Et al. (1993) “A protein alignment scoring system sensitive at all evolutionary distances “J. Mol. Evol. 36 (1993), 290-300.
Altschul et al. (1997) “Gapped BLAST and PSI-BLAST: a new generation of protein database search programs.” Nucleic Acids Research 25 3389-3402.
Brutlag et al. (1990) Improved sensitivity of biological sequence database searches. Computer Applications in the Biosciences 6: 237-245.
Henikoff and Henikoff (1992) “Amino acid substitution matrices from protein blocks” PNAS 89(22):10915-9Altschul et al. (1990) “Basic local alignment search tool.” J. Mol. Biol. 215, 403-410.
Thompson et al. (1994) “CLUSTAL W: improving the sensitivity of progressive multiple sequence alignment through sequence weighting, position-specific gap penalties and weight matrix choice” Nucleic Acids Research, 1994, Vol. 22, No. 22 4673-4680.

1. A method for detecting one or more of SARS-CoV 2 virus omicron, alpha, beta, delta and/or gamma in a sample, the method comprising
(i) sequencing a nucleic acid comprising a nucleic acid having at least 80% identity to a nucleic acid corresponding to positions 76 to 645 of the nucleic acid as set forth in SEQ ID NO. 1, wherein the sequenced nucleic acid has a length of at most 1500 nucleotides; and wherein the nucleic acid has been derived from the sample; and
(ii) identifying from the sequenced nucleic acid
(iia) one or more nucleotide replacement(s) at positions corresponding to positions 76, 77, 78, 238, 239, 240, 412, 413, 414, 424, 425, 426, 466, 467, 468, 568, 569, 570, 643, 644, and 645 of the nucleic acid as set forth in SEQ ID NO. 1; and/or
(iib) one or more nucleotide deletion(s) in the sequenced nucleic acid at positions corresponding to positions 205, 206, 207, 208, 209, 210, 430, 431, 432, 469, 470, 471, 472, 473 and 474 of the nucleic acid as set forth in SEQ ID NO. 1; and
(iii) correlating the one or more nucleotide replacement(s) and/or one or more nucleotide deletion(s) with the presence of one or more of SARS-CoV 2 virus omicron, alpha, beta, delta and/or gamma.

2. The method of claim 1, wherein the sample is a biological sample or an environmental sample.

3. The method of claim 1, wherein the detecting is diagnosing a subject with one or more of SARS-CoV 2 virus omicron, alpha, beta, delta and/or gamma, and wherein the nucleic acid has been derived from a biological sample that has been obtained from the subject.

4. The method of claim 1, wherein the SARS-CoV 2 virus omicron comprises nucleotide deletions at positions corresponding to positions 205, 206, 207, 208, 209, and 210 of SEQ ID NO. 1 and one or more nucleotide replacement(s) at positions corresponding to positions 424, 425 and/or 426 of SEQ ID NO.1, and/or
wherein the SARS-CoV 2 virus omicron comprises amino acid deletions at positions corresponding to positions 69 and 70 of SEQ ID NO. 2 and an amino acid replacement from G to D at a position corresponding to position 142 of SEQ ID NO.2.

5. The method of claim 1, wherein the SARS-CoV 2 virus alpha comprises nucleotide deletions at positions corresponding to positions 205, 206, 207, 208, 209, 210, 430, 431 and 432 of SEQ ID NO. 1, and/or
wherein the SARS-CoV 2 virus alpha comprises amino acid deletions at positions corresponding to positions 69, 70 and 144 of SEQ ID NO. 2.

6. The method of claim 1, wherein the SARS-CoV 2 virus beta comprises one or more nucleotide replacement(s) at positions corresponding to positions 238, 239 and 240 of SEQ ID NO. 1 and one or more nucleotide replacement(s) at positions corresponding to positions 643, 644 and 645 of SEQ ID NO. 1 and/or
wherein the SARS-CoV 2 virus beta comprises amino acid replacements from D to A at a position corresponding to position 80 of SEQ ID NO. 2 and from D to G at a position corresponding to positions 80 and 215 of SEQ ID NO. 2.

7. The method of claim 1, wherein the SARS-CoV 2 virus delta comprises nucleotide deletions at positions corresponding to positions 469, 470, 471, 472, 473, and 474 of SEQ ID NO. 1, one or more nucleotide replacement(s) at positions corresponding to position 424, 425 and/or 426 of SEQ ID NO. 1, and one or more nucleotide replacement(s) at positions corresponding to positions 466, 467 and/or 468 of SEQ ID NO. 1, and/or
wherein the SARS-CoV 2 virus delta comprises amino acid deletions at positions corresponding to positions 157 and 158 of SEQ ID NO. 2, an amino acid replacement from G to D at a position corresponding to position 142 of SEQ ID NO. 2, and an amino acid replacement from E to G at a position corresponding to position 156 of SEQ ID NO. 2.

8. The method of claim 1, wherein the SARS-CoV 2 virus gamma comprises one or more nucleotide replacement(s) at positions corresponding to positions 76, 77 and/or 78 of SEQ ID NO. 1, one or more nucleotide replacement(s) at positions corresponding to position 412, 413 and/or 414 of SEQ ID NO. 1, and one or more nucleotide replacement(s) at positions corresponding to positions 568, 569 and/or 570 of SEQ ID NO.1, and/or
wherein the SARS-CoV 2 virus gamma comprises amino acid replacement from P to S at a position corresponding to position 26 of SEQ ID NO. 2, an amino acid replacement from D to Y at a position corresponding to position 138 of SEQ ID NO. 2, and an amino acid replacement from R to S at a position corresponding to position 190 of SEQ ID NO.2.

9. The method of claim 1, wherein the sequenced nucleic acid has a length of at most 1400 nucleotides, at most 1300 nucleotides, at most 1200 nucleotides, at most 1100 nucleotides, at most 1000 nucleotides, at most 900 nucleotides, at most 800 nucleotides, at most 700 nucleotides or at most 600 nucleotides or less.

10. The method of claim 1, wherein the method further comprises a step
(a) extracting RNA nucleic acid from a sample.

11. The method of claim 1, wherein the method further comprises a step
(b) converting the RNA nucleic acid into the corresponding cDNA nucleic acid.

12. The method of claim 1, wherein the method further comprises step
(c) amplifying a cDNA nucleic acid having at least 80% identity to a nucleic acid corresponding to positions 76 to 645 of the nucleotide (cDNA) sequence as set forth in SEQ ID NO. 1, wherein the amplified cDNA nucleic acid has a length of at most 1500 nucleotides.

13. The method of claim 1, wherein the nucleic acid that is sequenced in step (i) is the cDNA obtained in step (c).

14. The method of claim 1, wherein the sequencing of step (i) and/or the amplification of step (c) comprises the use of an oligonucleotide of SEQ ID NO. 28 and an oligonucleotide of SEQ ID NO. 29.

15. The method of claim 1, wherein the method further comprises a step
(e) comparing the sequenced nucleic acid to the nucleic acid sequence of positions 76 to 645 as set forth in SEQ ID NO. 1; and/or
(e) comparing the amino acid sequence to the amino acid sequence of positions 26 to 215 as set forth in SEQ ID NO. 2.

16. The method of claim 1, wherein the method further comprises a step
(d) converting the sequenced nucleic acid into the corresponding amino acid sequence.

17. The method of claim 13, wherein the corresponding amino acid sequence corresponds to positions 26 to 215 of the sequence as set forth in SEQ ID NO. 2.

18. The method of claim 1, wherein the method further comprises step
(f) correlating the one or more nucleotide replacement(s) and/or one or more nucleotide deletion(s) and/or the one or more amino acid replacement(s) and/or one or more amino acid deletion(s) with
(x) omicron, when nucleotide deletions at positions corresponding to positions 205, 206, 207, 208, 209, and 210 of SEQ ID NO. 1 and one or more nucleotide replacements at positions corresponding to positions 424, 425 and/or 426 of SEQ ID NO.1 are detected; and/or
when amino acid deletions at positions corresponding to positions 69 and 70 of SEQ ID NO. 2 and an amino acid replacement from G to D at a position corresponding to position 142 of SEQ ID NO. 2 are detected;
(xi) alpha, when nucleotide deletions at positions corresponding to positions 205, 206, 207, 208, 209, 210, 430, 431 and 432 of SEQ ID NO. 1 are detected;
(xii) beta, when one or more nucleotide replacement(s) at positions corresponding to positions 238, 239 and 240 of SEQ ID NO. 1 and one or more nucleotide replacement(s) at positions corresponding to positions 643, 644 and 645 of SEQ ID NO. 1 are detected; and/or
when amino acid replacements from D to A at a position corresponding to position 80 of SEQ ID No. 2 and from D to G at a position corresponding to position 215 of SEQ ID NO. 2 are detected;
(xiii) delta, when nucleotide deletions at positions corresponding to positions 469, 470, 471, 472, 473, and 474 of SEQ ID NO. 1, one or more nucleotide replacements at positions corresponding to positions 424, 425 and/or 426 of SEQ ID NO. 1, and one or more nucleotide replacement(s) at positions corresponding to positions 466, 467 and/or 468 of SEQ ID NO. 1 are detected; and/or
when amino acid deletions at positions corresponding to positions 157 and 158 of SEQ ID NO. 2, an amino acid replacement from G to D at a position corresponding to position 142 of SEQ ID NO. 2, and an amino acid replacement from E to G at a position corresponding to position 156 of SEQ ID NO. 2 are detected;
(xiv) gamma, when one or more nucleotide replacement(s) at positions corresponding to positions 76, 77 and/or 78 of SEQ ID NO. 1, one or more nucleotide replacement(s) at positions corresponding to position 412, 413 and/or 414 of SEQ ID NO. 1, and one or more nucleotide replacement(s) at positions corresponding to positions 568, 569 and/or 570 of SEQ ID NO.1 are detected, and/or
when an amino acid replacement from P to S at a position corresponding to position 26 of SEQ ID NO. 2, an amino acid replacement from D to Y at a position corresponding to position 138 of SEQ ID NO. 2, and an amino acid replacement from R to S at a position corresponding to position 190 of SEQ ID NO.2 are detected.

19. A kit comprising components for preparing a nucleic acid from a sample that has SARS-CoV 2 virus RNA, wherein the nucleic acid has at least 80% identity to a nucleic acid corresponding to positions 76 to 645 of nucleic acid of SEQ ID NO. 1; wherein the nucleic acid has a length of at most 1500 nucleotides.

20. The kit of claim 19, wherein the kit comprises an oligonucleotide of SEQ ID NO. 28 and/or an oligonucleotide of SEQ ID NO. 29.


SEQUENCE LISTING (32 sequences)

SEQ ID NO: 1 - DNA, 3822 bp, synthetic construct
  source 1..3822: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 2 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 2070 bp, synthetic construct
  source 1..2070: mol_type = other DNA
catgcaaattacatattttggaggaatacaaatccaattcagttgtcttcctattctttatttgacatgagtaaatttccccttaaattagtacgtttaatgtataaaacctccttatgtttaggttaagtcaacagaaggataagaaataaactgtactcatttaaaggggaatttaataggggtactgctgttatgtctttaaaagaaggtcaaatcaatgatatgattttatctcttcttagtaaaggtagacttataattagagaatccccatgacgacaatacagaaattttcttccagtttagttactatactaaaatagagaagaatcatttccatctgaatattaatctcttaacaacagagttgttatttctagtgatgttcttgttaacaactaaacgaacaatgtttgtttttcttgttttattgccactagtctctagttgttgtctcaacaataaagatcactacaagaacaattgttgatttgcttgttacaaacaaaaagaacaaaataacggtgatcagagatctcagtgtgttaatcttacaaccagaactcaattaccccctgcatacactaattctttcacacgtggtgtttattaccctgacaaagttttagtcacacaattagaatgttggtcttgagttaatgggggacgtatgtgattaagaaagtgtgcaccacaaataatgggactgtttcaaaacagatcctcagttttacattcaactcaggacttgttcttacctttcttttccaatgttacttggttccatgctatacatgtctctgggacgtctaggagtcaaaatgtaagttgagtcctgaacaagaatggaaagaaaaggttacaatgaaccaaggtacgatatgtacagagaccctgcaatggtactaagaggtttgataaccctgtcctaccatttaatgatggtgtttattttgcttccactgagaagtctaacataataagagggttaccatgattctccaaactattgggacaggatggtaaattactaccacaaataaaacgaaggtgactcttcagattgtattattctccctggatttttggtactactttagattcgaagacccagtccctacttattgttaataacgctactaatgttgttattaaagtctgtgaattgacctaaaaaccatgatgaaatctaagcttctgggtcagggatgaataacaattattgcgatgattacaacaataatttcagacacttaatcaattttgtaatgatccatttttgggtgtttattaccacaaaaacaacaaaagttggatggaaagtgagttcagagtttattctagtgcagttaaaacattactaggtaaaaacccacaaataatggtgtttttgttgttttcaacctacctttcactcaagtctcaaataagatcacggaataattgcacttttgaatatgtctctcagccttttcttatggaccttgaaggaaaacagggtaatttcaaaaatcttagggaatttgtcttattaacgtgaaaacttatacagagagtcggaaaagaatacctggaacttccttttgtcccattaaagtttttagaatcccttaaacagtttaagaatattgatggttattttaaaatatattctaagcacacgcctattaatttagtgcgtgatctccctcagggtttttcggctttcaaattcttataactaccaataaaattttatataagattcgtgtgcggataattaaatcacgcactagagggagtcccaaaaagccgaaaagaaccattggtagatttgccaataggtattaacatcactaggtttcaaactttacttgctttacatagaagttatttgactcctggtgatcttggtaaccatctaaacggttatccataattgtagtgatccaaagtttgaaatgaacgaaatgtatcttcaataaactgaggaccactttcttcttcaggttggacagctggtgctgcagcttattatgtgggttatcttcaacctaggacttttctattaaaatataatgaaaatgg

SEQ ID NO: 4 - DNA, 25 bp, synthetic construct
  source 1..25: mol_type = other DNA
tgattggtgattgtgcaactgtaca

SEQ ID NO: 5 - DNA, 29 bp, synthetic construct
  source 1..29: mol_type = other DNA
tgttcgtttagttgttaacaagaacatca

SEQ ID NO: 6 - DNA, 26 bp, synthetic construct
  source 1..26: mol_type = other DNA
aggggtactgctgttatgtctttaaa

SEQ ID NO: 7 - DNA, 22 bp, synthetic construct
  source 1..22: mol_type = other DNA
aagtagggactgggtcttcgaa

SEQ ID NO: 8 - DNA, 23 bp, synthetic construct
  source 1..23: mol_type = other DNA
tgggaccaatggtactaagaggt

SEQ ID NO: 9 - DNA, 22 bp, synthetic construct
  source 1..22: mol_type = other DNA
accagctgtccaacctgaagaa

SEQ ID NO: 10 - DNA, 23 bp, synthetic construct
  source 1..23: mol_type = other DNA
gtgatctccctcagggtttttcg

SEQ ID NO: 11 - DNA, 26 bp, synthetic construct
  source 1..26: mol_type = other DNA
acttaaaagtggaaaatgatgcggaa

SEQ ID NO: 12 - DNA, 22 bp, synthetic construct
  source 1..22: mol_type = other DNA
acttgtgcccttttggtgaagt

SEQ ID NO: 13 - DNA, 22 bp, synthetic construct
  source 1..22: mol_type = other DNA
tgctggtgcatgtagaagttca

SEQ ID NO: 14 - DNA, 22 bp, synthetic construct
  source 1..22: mol_type = other DNA
ccggtagcacaccttgtaatgg

SEQ ID NO: 15 - DNA, 22 bp, synthetic construct
  source 1..22: mol_type = other DNA
cccctattaaacagcctgcacg

SEQ ID NO: 16 - DNA, 24 bp, synthetic construct
  source 1..24: mol_type = other DNA
accaggttgctgttctttatcagg

SEQ ID NO: 17 - DNA, 24 bp, synthetic construct
  source 1..24: mol_type = other DNA
cagctattccagttaaagcacggt

SEQ ID NO: 18 - DNA, 27 bp, synthetic construct
  source 1..27: mol_type = other DNA
aattctaccagtgtctatgaccaagac

SEQ ID NO: 19 - DNA, 22 bp, synthetic construct
  source 1..22: mol_type = other DNA
gcaccaaaggtccaaccagaag

SEQ ID NO: 20 - DNA, 23 bp, synthetic construct
  source 1..23: mol_type = other DNA
gctgctagagacctcatttgtgc

SEQ ID NO: 21 - DNA, 22 bp, synthetic construct
  source 1..22: mol_type = other DNA
aagctctgatttctgcagctct

SEQ ID NO: 22 - DNA, 28 bp, synthetic construct
  source 1..28: mol_type = other DNA
aaatgatatcctttcacgtcttgacaaa

SEQ ID NO: 23 - DNA, 25 bp, synthetic construct
  source 1..25: mol_type = other DNA
tgagtctaattcaggttgcaaagga

SEQ ID NO: 24 - DNA, 23 bp, synthetic construct
  source 1..23: mol_type = other DNA
gcacacactggtttgtaacacaa

SEQ ID NO: 25 - DNA, 22 bp, synthetic construct
  source 1..22: mol_type = other DNA
tttgactcctttgagcactggc

SEQ ID NO: 26 - DNA, 26 bp, synthetic construct
  source 1..26: mol_type = other DNA
ctaggttttatagctggcttgattgc

SEQ ID NO: 27 - DNA, 22 bp, synthetic construct
  source 1..22: mol_type = other DNA
catttccagcaaagccaaagcc

SEQ ID NO: 28 - DNA, 26 bp, synthetic construct
  source 1..26: mol_type = other DNA
aggggtactgctgttatgtctttaaa

SEQ ID NO: 29 - DNA, 22 bp, synthetic construct
  source 1..22: mol_type = other DNA
accagctgtccaacctgaagaa

SEQ ID NO: 30 - DNA, 20 bp, synthetic construct
  source 1..20: mol_type = other DNA
tcttggaggttccgtggcta

SEQ ID NO: 31 - DNA, 20 bp, synthetic construct
  source 1..20: mol_type = other DNA
tatcttggcaaaccacgcga

SEQ ID NO: 32 - DNA, 21 bp, synthetic construct
  source 1..21: mol_type = other DNA
gatgcaaatctggtggcgtta